(19) United States
(12) Patent Application Publication
(10) Pub. No.: US 2010/0266640 A1
(43) Pub. Date: Oct. 21, 2010

(54) Plant-Derived Cholera and Malaria Vaccine

(76) Inventor: Henry Daniell, Winter Park, FL (US)

Correspondence Address: Timothy H. Van Dyke, 390 No. Orange Avenue, Suite 2500, Orlando, FL, 32801, US

(21) Appl. No.: 12/763,562

(22) Filed: Apr. 20, 2010

Related U.S. Application Data
(60) Provisional application No. 61/170,969, filed on Apr. 20, 2009.

(51) Int. Cl. A61K 39/106; A61P 31/04; A61P 33/06; A01H 5/00

(52) U.S. Cl. 424/261.1; 800/298

(57) ABSTRACT

Described herein are methods for simultaneously immunizing a subject against Cholera and Malarial infection. Specifically exemplified herein are methods that involve administering compositions comprising a CTB-AMA1 or CTB-MSP1 derived from plants having plastids transformed to express such conjugates.

### CROSS-REFERENCE TO RELATED APPLICATIONS

This application is related to U.S. Provisional Application 61/170,969; filed Apr. 20, 2009 to which priority is claimed. This application is incorporated herein in its entirety by this reference.

### BACKGROUND

Cholera is one among the top three diseases listed by the World Health Organization (WHO) and the mortality rate is estimated to be 100,000-150,000 deaths annually₁ and remains the most devastating diarrheal disease, especially under severe weather conditions that increase water pollution. More recent cholera outbreaks have been reported in Kenya, Nigeria and Vietnam. Rapidly waning immunity with infection both from human and environmental sources has been recently reported (King, A. A., Ionides, E. L., Pascual, M. & Bouma, M. J. Inapparent infections and cholera dynamics. Nature 454, 877-880 (2008)). However, only one internationally licensed cholera vaccine is available but this remains prohibitively expensive for routine use in cholera-endemic areas in developing countries (Mahalanabis, D. et al. A randomized, placebo-controlled trial of the bivalent killed, wholecell, oral cholera vaccine in adults and children in a cholera endemic area in Kolkata, India. PLoS. ONE. 3, e2323 (2008)), especially at times of outbreak. Also, with the current cholera vaccine, immunity is lost in children within three years and adults are not fully protected (Olsson, L. & Parment, P. A. Present and future cholera vaccines. Expert. Rev. Vaccines 5, 751-752 (2006)). Oral cholera vaccines are ideal for developing countries (Lopez, A. L., Clemens, J. D., Deen, J. & Jodar, L. Cholera vaccines for the developing world. Hum. Vaccin 4, 165-169 (2008).

Malaria is also a devastating global health problem in tropical and subtropical areas of over 100 countries. Plasmodium falciparum is the most virulent species with approximately 500 million cases, one million deaths annually and more than two billion people are at risk for malaria (Greenwood B M, Bojang K, Whitty C J, Targett G A (2005) Malaria. Lancet 365:1487-1498; Langhorne J, Ndungu F M, Sponaas A M, Marsh K (2008) Immunity to malaria: more questions than answers. Nat Immunol 9:725-732. There are many challenges in developing a durable vaccine against malaria because of the complexity of antigens, high polymorphism among parasitic proteins, lack of appropriate animal model, high cost of vaccine development and delivery (Aide P, Bassat Q, Alonso P L (2007) Towards an effective malaria vaccine. Arch Dis Child 92:476-479).

### BRIEF DESCRIPTION OF THE DRAWINGS

FIG. 1 Regeneration of transplastomic plants and confirmation of transgene integration. (a-c) First, second and third rounds of regeneration. (d) Confirmation of maternal inheritance by germinating seeds in MS liquid medium containing spectinomycin 50 mg/L (UT, untransformed; T, transplastomic line). (e, f) Schematic representation of the lettuce chloroplast genome flanking sequence used for homologous recombination, probe DNA sequence (1.13 kb) and lettuce chloroplast transformation vector including the transgene cassette, integration site and anticipated products of the transgenic lines. (g, h) PCR analysis of the transgenic lines using 16SF/3M and 5P/2M primer pairs (UT, untransformed; T1 to T3, transgenic lines; P, positive control; M, 1 kb plus DNA ladder). Southern blots hybridized with the flanking sequence (i) and CTB (j) probes (T1 to T3, transplastomic lines, UT, untransformed).

FIG. 2 Expression of CTB via the lettuce chloroplast genome. Western blots for evaluation of CTB expression under reducing (a) and non-reducing (b) conditions. M, protein marker; 1, untransformed; 2, 4, 6 and 8 blank; 3, 5 and 7, transgenic lines; Std, purified CTB standard 30 ng. (c) ELISA showing expression levels of CTB in the total soluble protein (TSP) under normal conditions of illumination in the green house. (d) GM-1 ganglioside binding assay: T1 to T3, transgenic lines; UT, untransformed.

FIG. 3 CHO elongation assays. (a) Pooled sera of immunized/control mice were neutralized with 50 ng of CT and then was added to the CHO cell culture as described in materials and methods. The conditions are as follows: A: RPMI, B: CT (50 ng/ml), C: UT, D: SQV, E: ORVCTB and F: Untreated cells. (b) Reversal of CHO morphological changes, 50% of the supernatant was replaced with fresh media. UC=untransformed

FIG. 4 Evaluation of immunoglobulins and cholera toxin (CT) challenge. (a) CT challenge in control and vaccinated mice. CT (1.5 μg of body weight) was challenged orally for 14 hrs. Representative intestinal samples are shown from the following groups. A: Control mouse gavaged with untransformed leaf (ORV-UT, n=5), B, C and D are ADJ (n=5), SQV (n=9) and ORV-CTB mice (n=10). (b) each point represents intestinal water content (μl) of individual mice in different groups after CT challenge (One-way ANOVA, p<0.0001). (c) CTB- antigen specific serum and intestinal IgA in different groups of mice measured by ELISA. (d) sera of SQV and ORV-CTB mice were subjected to antigen-specific CTB-Igs ELISAs as shown in each panel. Top row shows CTB-IgG1 and -IgG2a titers; middle row shows CTB-IgG2b and IgG3 titers; the bottom row shows serum CTB-IgM titers before and after CT challenge. Data represent one of at least 3-5 independent experiments for any given Ig. (e) determination of effectiveness of numbers of boosters to generate antigen-specific serum IgA in oral gavage with transgenic leaf materials. Ten week old mice were boosted subcutaneously (until 189 days) or orally (until 220 days). Sera were collected until 197 days post-immunization. Ctrl un-chal=control un-challenged; AJV=adjuvant vaccinated; SQV=subcutaneous immunization; ORV=oral immunization.

FIG. 5 Flow Cytometry. Flow cytometry analyses were performed on fresh single-cell suspension of splenocytesobtained from unimmunized/control (n=3), unimmunized/CT challenged (n=2), SQV (n=4) and ORV-CTB mice (n=5) after CT challenge as described in details in materials and methods. Cell surface staining was performed using anti-mouse CD4, CD25, CD127, CD11c, CD80 and biotinconjugated MHC II and then stained with streptavidin conjugated PerCP (BD Bioscience). Purified rat anti-mouse CD16/CD32 was used for 10 min to block Fc receptor before initiation of cell surface staining. Intra-cellular staining of Foxp3, IL-4, IL-10 and IFNγ was performed using FoXp3 intra cellular staining kit (eBioscience) according to instructions provided by manufacturer. Splenic dendritic cells were stained as described earlier and flow cytometry was performed as described above and 30,000 events were acquired. Splenocytes are gated on CD4+ T-cells and CD11c+high splenic cells.

FIG. 6. Schematic presentation of the lettuce and tobacco chloroplast constructs. Schematic representation of the lettuce and tobacco chloroplast genome flanking sequences used for homologous recombination, probe DNA sequence and chloroplast transformation vectors including the transgene cassettes for CTB, CTB-AMA1, CTB-MSP1 integration sites and anticipated products of the transplatomic lines in Southern blots. [symbol] represents lettuce 16s ribosomal operon promoter; [symbol] represents lettuce 3′ rbcL; [symbol] represents lettuce psbA promoter including 5′ untranslated region (UTR); [symbol] represents lettuce psbA 3′UTR; [symbol] represents tobacco psbA promoter including 5′UTR; [symbol] represents tobacco psbA 3′UTR; [symbol] represents tobacco 16s ribosomal operon promoter.

FIG. 7. Southern blots analyses of transgenic plants. Southern blots hybridized with the lettuce and tobacco flanking sequence probes and CTB. (A) Tobacco transplastomic lines. Lane 1: untransformed (4.1 kb), lane 2: homoplasmic CTB- MSP1 (6.5 kb), and lane 3: homoplasmic CTB-AMA1 (6.6 kb). (B) Lettuce transplastomic lines. Lane 1: untransformed (9.1 kb), lane 2: blank, lane 3 & 4: homoplasmic CTB-AMA1 (11.6 kb), Lane 5: untransformed, lane 6 & 7: homoplasmic CTB-MSP1 (11.5 kb). (C) Lettuce CTB transplastomic lines, lanes 1-3: homoplasmic (5.23 kb), lane 4: untransformed (3.13 kb). (D) lettuce CTB transplastomic lines probed with CTB. Lanes 1 to 3: transplastomic, lane 4: untransformed.

FIG. 8. Expression of vaccine antigens in transgenic chloroplasts. Western blots for evaluation of expression in chloroplasts of (A) CTB-AMA1 in tobacco: Lane 1: untransformed extract, lane 2: monomeric 11.6 kDa CTB protein, lane 3: pellet, lane 4: supernatant. (B) CTB-MSP1 in tobacco: Lane 1: untrasformed, lane 2: CTB MSP-1 expression in E. coli, lane 3: blank, lane 4: pellet, lane 5: supernatant. (C) CTB-AMA1 expression in lettuce. M: protein marker, lanes 1 & 3: 11.6 kDa monomeric CTB protein standard, lane 2: untransformed, lanes 4 & 5: CTB-AMA1 expression in lettuce (homogenate). Lane 6: CTB-AMA1 expression in tobacco (homogenate). (D) CTB-MSP1 expression in lettuce. Lanes 1, 2 & 3: monomeric CTB protein standard (50 ng, 100 ng and 200 ng, respectively), lanes 4 & 5: lettuce transgenic lines expressing CTB-MSP1 (homogenate), lane 6: blank, lane 7: tobacco transgenic line expressing CTB-MSP1. (E) CTB expression in lettuce under reducing and (F) non-reducing condition. M: protein marker, lane 1: untransformed, lane 2, 4, 6 and 8: blank, lane 3, 5 and 7: lettuce transgenic lines, lane 9: purified CTB standard (30 ng). (G) GM-1 ganglioside binding assay: T1 to T3, transgenic lines; UT, untransformed.

FIG. 9. Enrichment of Chloroplast-Derived CTB Malarial Antigens. (A) CTB FC AMA1 protein was extracted from transformed leaves and the crude extract was subjected to Talon Superflow Metal Affinity Resin and analyzed. Molecular size standards are indicated in lanes 1 & 7. Lanes 2-6: reduced and lanes 8-12: non-reduced conditions of CTB FC AMA1 protein enrichment was observed by using a gradient gel (4-12%) and gel electrophoresis. The following fragments were visualized: lanes 2, & 8: untransformed, lanes 3 & 9: lysate, lanes 4 & 10: flow through, lanes 5 & 11: wash, and lanes 6 & 12: enriched protein. (B) Immunoblot analysis of tobacco CTB FC AMA1, lanes 1-4: CTB protein (1000, 500, 250, 125 ng, respectively), lane 5: protein marker, lanes 6-9: eluted CTB FC AMA1 (1.5, 0.75, 0.375, 0.1875 μg, respectively). (C) Immunoblot analysis of tobacco CTB MSP1. Lanes 1-4.: CTB protein (1000, 500, 250, 125 ng respectively), lanes 5 -7: eluted CTB MSP1 (1.5, 0.75, 0.375 μg, respectively). Eluted proteins and CTB were subjected to densitometry to determine the enrichment of CTB FC AMA1 and CTB MSP1 to be administered to mice for subcutaneous injection.

FIG. 10. CHO elongation assays. (A) Pooled sera of immunized/control mice were neutralized with 50 ng of CT and then was added to the CHO cell culture as described in materials and methods. The conditions are as follows: 1: RPMI, 2: CT (50 ng/ml), 3: UT, 4: SQV, 5: ORV-CTB and 6: Untreated cells. (B) Reversal of CHO morphological changes, 50% of the supernatant was replaced with fresh media. UC=untransformed

FIG. 11. Evaluation of immunoglobulins and cholera toxin (CT) challenge. (A) CT challenge in control and vaccinated mice. CT (1.5 μg/g of body weight) was challenged orally for 14 hrs. Representative intestinal samples are shown from the following groups. 1: Control mouse gavaged with untransformed leaf (ORV-UT, n=5), 2, 3 and 4 are ADJ (n=5), SQV (n=9) and ORV-CTB mice (n=10). (B) Each point represents intestinal water content (μl) of individual mice in different groups after CT challenge (One-way ANOVA, p<0.0001). (C) CTB-antigen-specific serum and intestinal IgA in different groups of mice measured by ELISA. (D) sera of SQV and ORV-CTB mice were subjected to antigen-specific CTB-Igs ELISAs as shown in each panel. Top row shows CTB-IgG1 and -IgG2a titers; middle row shows CTB-IgG2b and IgG3 titers; the bottom row shows serum CTB-IgM titers before and after CT challenge. Data represent one of at least 3-5 independent experiments for any given Ig. (E) determination of effectiveness of numbers of boosters to generate antigen-specific serum IgA in oral gavage with transgenic leaf materials. Ten week old mice were boosted subcutaneously (until 189 days) or orally (until 220 days). Sera were collected until 197 days post-immunization. Ctrl un-chat=control un-challenged; AJV=adjuvant vaccinated; SQV=subcutaneous immunization; ORV=oral immunization.

FIG. 12. Cross-reactivity of antisera generated against transgenic malaria vaccine antigens. (A) Immunoblot analysis: 36.8 μg of cell-free parasite extracts from ring, trophozoite, and schizont stages were resolved on SDS-PAGE gels and were subjected to immunoblot analysis using diluted sera from immunized mice. Immune sera collected from immunized mice recognized the native 83-kDa AMA1 protein (lanes 1-3) and the native 190 kDa MSP-1 protein (lanes 4-6). The parasite stages analyzed from P. falciparum 3D7 culture were ring: lanes 1 & 4, trophozoite: lanes 2 & 5 and schizont: lanes 3 & 6. (B) Immunofluorescence analysis: P. falciparum 3D7 parasites were immunostained with anti-AMA1 (top row) and anti-MSP1 antibodies (lower row) from immunized mice. Panels 1 and 4 are differential interference contrast images, panels 2 and 5 are fluorescence images, and panel 3 and 6 are merge images of previous two panels. The AMA1 antibodies recognized the apical end of the parasite in the ring developmental stage of intraerythrocytic growth (1, 2 and 3). The MSP-1 sera from immunized mice (bottom row) detected the developing merozoites at the schizont stage of the parasitic growth (4, 5 and 6). Bar size=10 μm.

FIG. 13. Single-cells based analyses of immunized/control mice. Flow cytometry analyses were performed on fresh single-cell suspension of splenocytes obtained from unimmunized/control (n=3), unimmunized/CT challenged (n=2), SQV (n=4) and ORV-CTB mice (n=5) after CT challenge as described in details in materials and methods. Cell surface staining was performed using anti-mouse CD4, CD25, CD127, CD11c, CD80 and biotin-conjugated MHC II and then stained with streptavidin conjugated PerCP (BD Bioscience). Purified rat anti-mouse CD16/CD32 was used for 10 min to block Fc receptor before initiation of cell surface staining. Intra-cellular staining of Foxp3, IL-4, IL-10 and IFNγ was performed using Foxp3 intra cellular staining kit (eBioscience) according to instructions provided by manufacturer. Splenic dendritic cells were stained as described earlier and flow cytometry was performed as described above and 30,000 events were acquired. Splenocytes are gated on CD4⁺ T-cells and CD11c^{+high} splenic cells.

### DESCRIPTION

Embodiments of the present invention pertain to methods and materials for effectuating the simultaneous immunization of a subject against cholera and malarial infection. The invention stems from the development of a plastid expression system for a CTB polypeptide conjugated to a malarial antigen. In more specific embodiments, the present invention pertains plastid transformation vectors that are capable of transforming a plastid to express a CTB-apical membrane antigen 1 (AMA1) conjugate and/or CTB-merozoite surface protein-1 (MSP1) conjugate. According to certain embodiments, the invention pertains to a method that involves administering to the subject a composition comprising a CTB-malarial antigen conjugate derived from a chloroplast engineered to express said such conjugate, and, optionally, a plant remnant. In a more specific embodiment, the plant remnant is from a plant edible without cooking.

Simultaneous immunization as used herein refers to the dual immunization of a subject to both cholera and malaria infection by administering a composition comprising an immunogen sufficient to induce immunization for both.

The term “a plant edible without cooking” refers to a plant that is edible, i.e., edible without the need to be subjected to heat exceeding 120 deg F for more than 5 min. Examples of such plants include, but are not limited to, Lactuca sativa (lettuce), apple, berries such as strawberries and raspberries, citrus fruits, tomato, banana, carrot, celery, cauliflower; broccoli, collard greens, cucumber, muskmelon, watermelon, pepper, pear, grape, peach, radish and kale. In a specific embodiment, the edible plant is Lactuca sativa.

Edible plants that require cooking or some other processing are not excluded from the teachings herein.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

In another embodiment, the invention pertains to an administerable composition for vaccinating a subject against cholera and malaria. The composition comprises a therapeutically-effective amount of a CTB-malarial antigen conjugate polypeptide having been expressed by a plant and a plant remnant. In specific embodiments, the conjugate is CTB-AMA1 or CTB-MSP1. In alternative embodiments, the composition comprises both CTB-AMA1 and CTB-MSP1.

According to a further embodiment, the invention pertains to a stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for a CTB protein or variants thereof, and AMA1 or MSP1, or variants thereof, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

It is the inventor's belief that biopharmaceutical proteins expressed in plant cells should reduce their cost of production. Transformation of plant nuclear genomes has led to the expression of a number clinically important molecules in cell culture, organized tissue culture and in whole plants (Rigano and Walmsley, 2005). Common crop species such as potatoes, rice and tomatoes have been engineered to express many therapeutic proteins via the nuclear genomes of these plants (Ma et al., 2003).

One of the major limitations has been the ability in these systems to accumulate sufficient levels of protein either for purification or for oral delivery in minimally processed plant tissues. Integration of transgenes via the nuclear genome may have other disadvantages including transgene containment, gene silencing, and position effect. The chloroplast genetic engineering approach overcomes concerns of transgene containment, gene silencing and position effect, pleiotropic effects, and presence of antibiotic resistant genes or vector sequences in transformed genomes.

Methods, vectors, and compositions for transforming plants and plant cells are taught for example in WO 01/72959; WO 03/057834; and WO 04/005467. WO 01/64023 discusses use of marker free gene constructs.

Proteins expressed in accord with certain embodiments taught herein may be used in vivo by administration to a subject, human or animal in a variety of ways. The pharmaceutical compositions may be administered orally or parenterally, i.e., subcutaneously, intramuscularly or intravenously, though oral administration is preferred.

Oral compositions produced by embodiments of the present invention can be administrated by the consumption of the foodstuff that has been manufactured with the transgenic plant producing the plastid derived therapeutic protein. The edible part of the plant, or portion thereof, is used as a dietary component. The therapeutic compositions can be formulated in a classical manner using solid or liquid vehicles, diluents and additives appropriate to the desired mode of administration. Orally, the composition can be administered in the form of tablets, capsules, granules, powders and the like with at least one vehicle, e.g., starch, calcium carbonate, sucrose, lactose, gelatin, etc. The preparation may also be emulsified. The active immunogenic ingredient is often mixed with excipients which are pharmaceutically acceptable and compatible with the active ingredient. Suitable excipients are, e.g., water, saline, dextrose, glycerol, ethanol or the like and combination thereof In addition, if desired, the compositions may contain minor amounts of auxiliary substances such as wetting or emulsifying agents, pH buffering agents, or adjuvants. In a preferred embodiment the edible plant, juice, grain, leaves, tubers, stems, seeds, roots or other plant parts of the pharmaceutical producing transgenic plant is ingested by a human or an animal thus providing a very inexpensive means of treatment of or immunization against disease.

In a specific embodiment, plant material (e.g. lettuce material) comprising chloroplasts capable of expressing a CTB-malarial conjugate protein is homogenized and encapsulated. In one specific embodiment, an extract of the lettuce material is encapsulated. In an alternative embodiment, the lettuce material is powderized before encapsulation.

In alternative embodiments, the compositions may be provided with the juice of the transgenic plants for the convenience of administration. For said purpose, the plants to be transformed are preferably selected from the edible plants consisting of tomato, carrot and apple, among others, which are consumed usually in the form of juice.

According to another embodiment, the subject invention pertains to a transformed chloroplast genome that has been transformed with a vector comprising a heterologous gene that expresses a peptide as disclosed herein. Of particular present interest is a transformed chloroplast genome that has been transformed with a vector comprising a heterologous gene that expresses a CTB-malarial peptide fusion protein. In a related embodiment, the subject invention pertains to a plant comprising at least one cell transformed to express a peptide as disclosed herein. In alternative embodiments, the invention pertains to plants comprising at least one plastid transformed to express a CTB-AMA1 or CTB-MSP1 conjugate.

Reference to a CTB polypeptide sequence herein relates to the full length amino acid sequences as well as at least 12, 15, 25, 50, 75, 100, 125, 150, 175, 200, 225, 250 or 265 contiguous amino acids selected from such amino acid sequences, or biologically active variants thereof. See Sanchez and Holmgren, PNAS 86:481-485 (1989) for polynucleotide and polypeptide sequences of CTB. See Bai et al., PNAS 102:12736-12741 (2005) for sequence information on AMA1 and structural features of same. See U.S. Pat. No. 6,933,130 for sequence information of MSP1.

Variants which are biologically active, refer to those, in the case of oral tolerance, that activate T-cells and/or induce a Th2 cell response, characterized by the upregulation of immunosuppressive cytokines (such as IL10 and IL4) and serum antibodies (such as IgG1), or, in the case of desiring the native function of the protein, is a variant which maintains the native function of the protein. Preferably, naturally or non-naturally occurring polypeptide variants have amino acid sequences which are at least about 55, 60, 65, or 70, preferably about 75, 80, 85, 90, 96, 96, or 98% identical to the full-length amino acid sequence or a fragment thereof. Percent identity between a putative polypeptide variant and a full length amino acid sequence is determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active CTB-malarial antigen polypeptide can readily be determined by assaying for native activity, as described for example, in the specific Examples, below.

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀ [Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

According to another embodiment, the invention pertains to a method of producing a CTB-AMA1 and/or CTB-MSP1 containing composition, the method including obtaining a stably transformed Lactuca sativa plant which includes a plastid stably transformed with an expression vector which has an expression cassette having, as operably linked components in the 5′ to the 3′ direction of translation, a promoter operative in a plastid, a selectable marker sequence, a heterologous polynucleotide sequence coding for comprising at least 70% identity to CTB protein, transcription termination functional in said plastid, and flanking each side of the expression cassette, flanking DNA sequences which are homologous to a DNA sequence of the target plastid genome, whereby stable integration of the heterologous coding sequence into the plastid genome of the target Lactuca sativa plant is facilitated through homologous recombination of the flanking sequence with the homologous sequences in the target plastid genome; and homogenizing material of said stably transformed Lactuca sativa plant to produce homogenized material.

According to another embodiment, the subject invention pertains to a pharmaceutical protein sample bioencapsulated in choroplasts of a plant cell. The chloroplasts have been modified to express the pharmaceutical protein. Protein is produced in the modified chloroplasts and barring rupture or some other disruptive stimulus, the protein is pooled and stored in the chloroplast. Thus the chloroplast acts as a protective encapsulation of the protein sample

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

### Example 1

### Oral and Injectable Chloroplast-Derived Cholera Vaccine Antigen Confer Long-Term Immunity and Protection Against Toxin Challenge

A. Characterization of Transplastomic Lettuce Expressing CTB

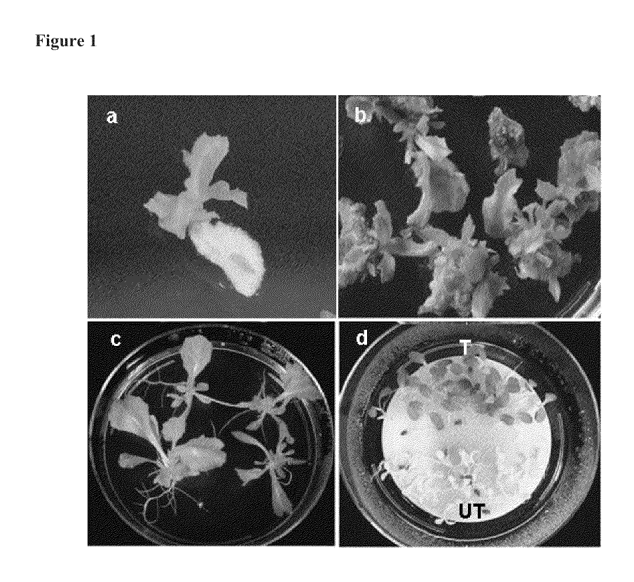

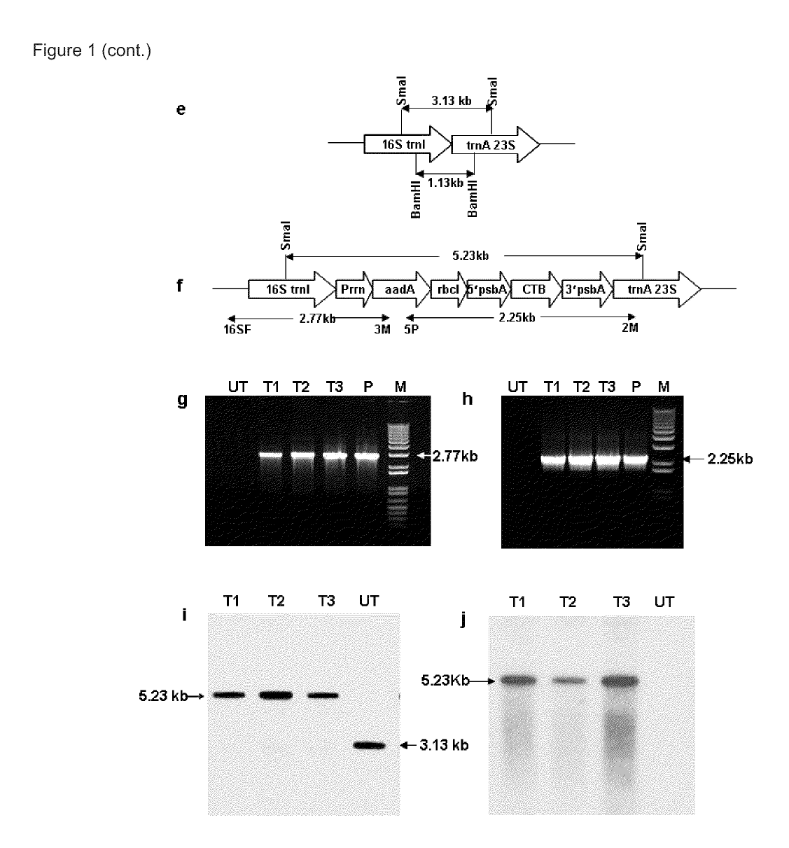

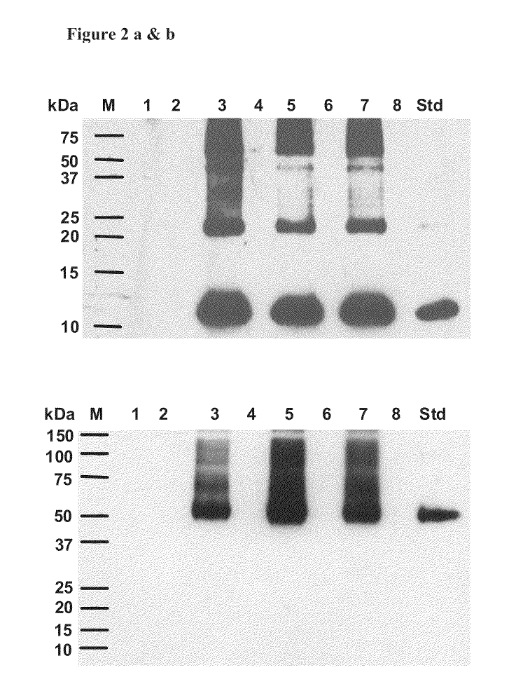

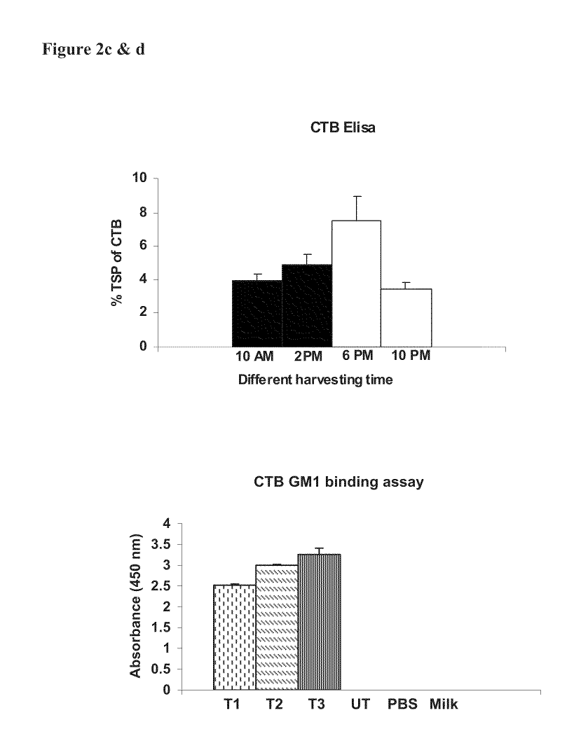

The lettuce chloroplast transformation vector pLsDV CTB was constructed as previously described using standard molecular biology protocols (Verma, D., Samson, N. P., Koya, V. & Daniell, H. A protocol for expression of foreign genes in chloroplasts. Nat. Protoc. 3, 739-758 (2008)). In this construct, 16S/trnI and trnA/23S genes were used as flanking sequences for homologous recombination with the native chloroplast genome. Transplastomic lettuce plants were obtained as described previously (Kanamoto, H. et al. Efficient and stable transformation of Lactuca sativa L. cv. Cisco (lettuce) plastids. Transgenic Res. 15, 205-217 (2006); Ruhlman, T., Ahangari, R., Devine, A., Samsam, M. & Daniell, H. Expression of cholera toxin B-proinsulin fusion protein in lettuce and tobacco chloroplasts—oral administration protects against development of insulitis in non-obese diabetic mice. Plant Biotechnol. J 5,495-510 (2007)). Green shoots emerged from the bombarded leaves after 3-6 weeks (FIG. 1a) and they were subjected to second (FIG. 1b) and third (FIG. 1c) rounds of regeneration to achieve homoplasmy. Transplastomic shoots were screened for transgene integration by PCR analysis using primers 16SF/3M and 5P/2M (FIG. 1f). The 16SF primer anneals to the native chloroplast genome upstream of the site of integration and 3M primer lands on the aadA gene producing a 2.77 kb PCR product. The 5P primer lands on the aadA gene and 2M lands on the trnA coding sequence, producing a 2.25 kb PCR product. All transformants showed respective PCR products, confirming site specific integration of the transgene cassette into the lettuce chloroplast genome (FIG. 1g, h). As shown in FIGS. 1i and 1j, site specific transgene integration into the chloroplast genome was confirmed by Southern blot analysis and all transgenic lines produced an expected fragment of 5.23 kb, while this was absent in untransformed lines. This result also confirms that all the transgenic lines achieved homoplasmy (FIG. 1i). Presence of CTB in transplastomes was confirmed by the CTB probe (FIG. 1j). T₁ seeds germinated and grew into uniformly green plants while untransformed plants were bleached on the selection medium indicating that the transgenic lines are maternally inherited to their progeny (FIG. 1d). Expression of CTB was confirmed by western blot analysis as illustrated in FIGS. 2a & b. The monomer, dimer and pentameric forms of CTB were observed in all transgenic lines under denatured condition and only the pentameric or larger forms were observed under non-reducing conditions. The expression levels of CTB in T₀ transplastomic lines reached up to 7.5% of total soluble protein (TSP) in mature leaves under normal growth conditions in the green house. Maximum level of CTB expression was observed from leaves harvested in the evening (FIG. 2c) because CTB is regulated by light.

B. GM₁ Binding of Chloroplast-Derived CTB

GM₁-ganglioside has been shown to be the receptor for CTB protein in vivo and a pentameric structure is required for binding to GM₁ receptor. To investigate functionality of chloroplast-derived CTB, we performed GM₁ binding ELISA assay. As illustrated in FIG. 2d, chloroplast-derived CTB is fully functional and binds to GM₁. These results confirm that the lettuce chloroplast derived CTB is properly folded to form pentamers, which is essential for GM₁-ganglioside receptor binding.

C. Sera of Immunized Mice Protects CHO Cells from Dehydration After CT Treatment

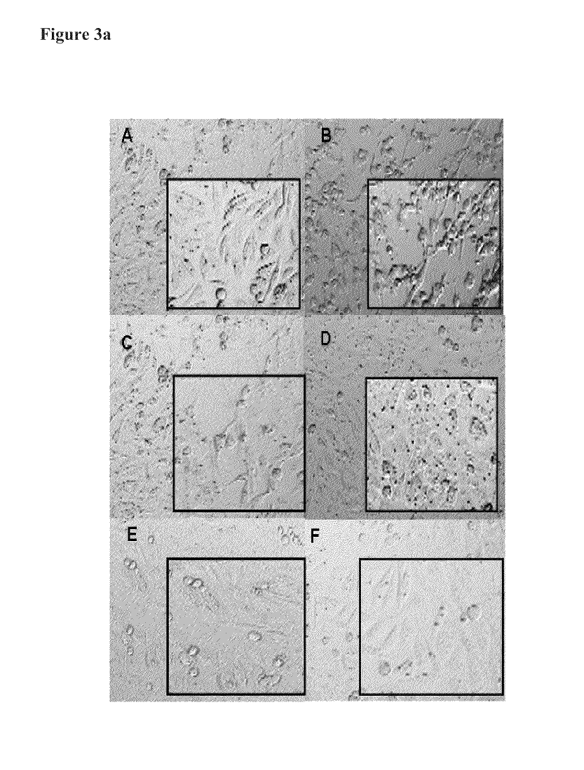

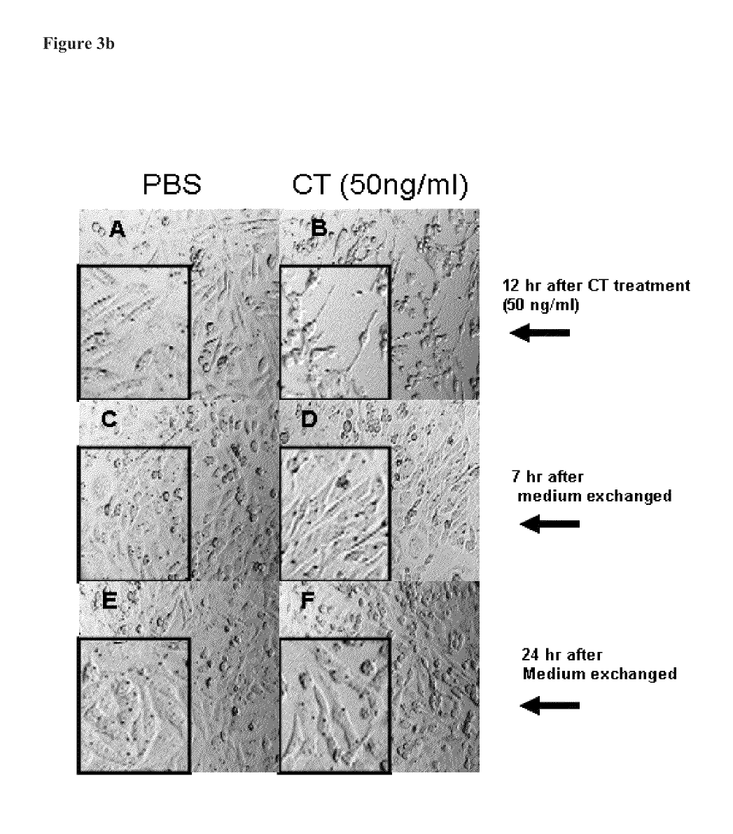

In order to examine the biological activity of antibodies induced by oral or subcutaneous administration of CTB, CHO cell elongation assay was performed with pooled sera of vaccinated and control mice as described elsewhere₁₂. Our data show that sera of immunized mice, regardless of route of immunization, protected morphological changes (elongation) due to dehydration in CHO cell culture (FIG. 3a). In contrast, CHO cells treated with sera of unimmunized control mice showed massive elongation. When cell viability was checked 12 hr after CT treatment, using trypan blue exclusion method, we were unable to find cell death (>5%) in all conditions tested, including the CT treated cells (positive controls). Based on this observation we reasoned that morphological changes in CHO cells is transient and can be reversed by toxin removal. To investigate this hypothesis, we replaced 50% of the cell culture supernatant containing CT with fresh media and examined cell morphology after 7, 12 and 24 hrs. As shown in FIG. 3b, almost 80% of the CHO cells recovered after 7 hrs and there was very little morphological difference between PBS treated (negative control) and CT treated cells after 12 hrs. CHO cells were indistinguishable with control PBS treated after 24 hrs (FIG. 3b). These data suggest that dehydration of CHO cells because of CT treatment is a transient state and cells can be reversed by CT removal within 7-24 hrs. To the best of our knowledge, reversibility of dehydration has not yet been described elsewhere.

D. Mechanism of Protection from Cholera Toxin Challenge

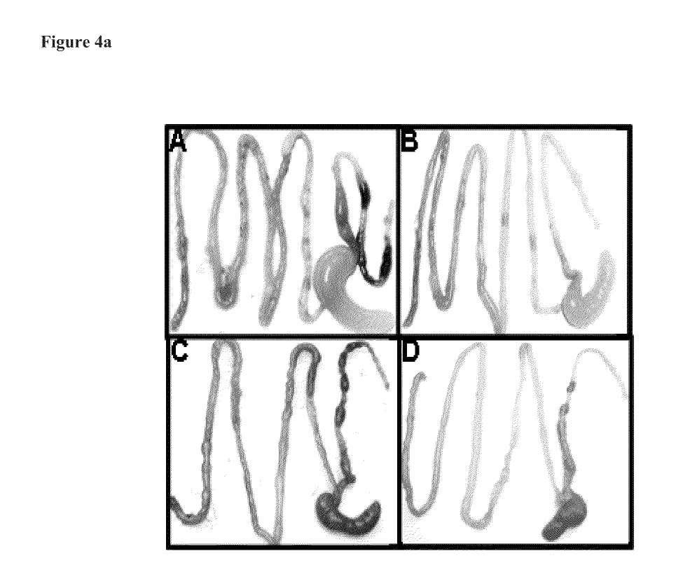

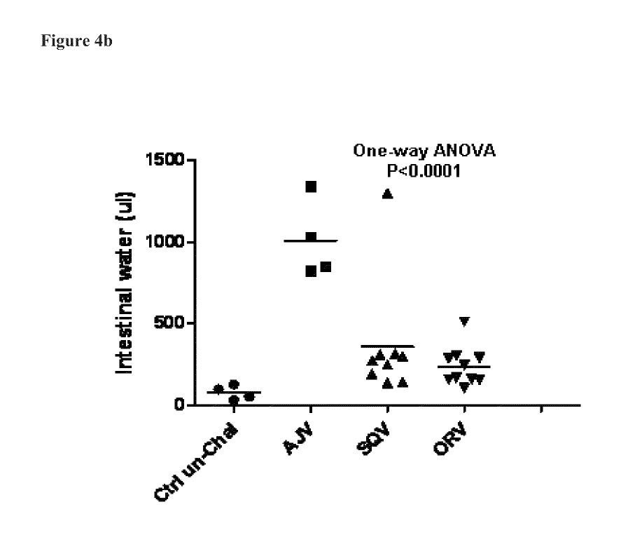

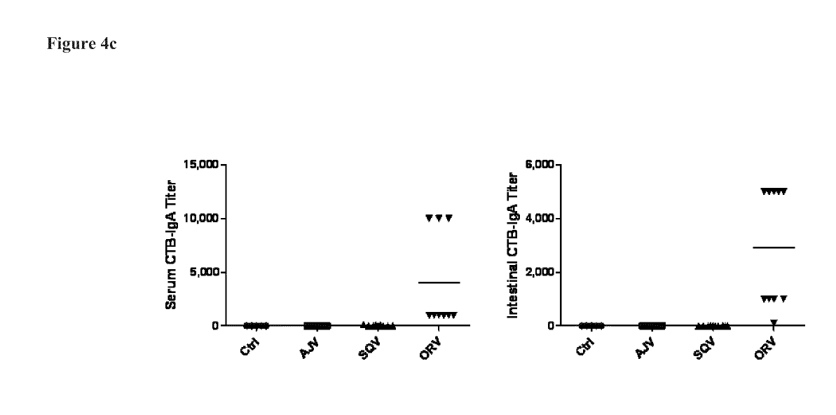

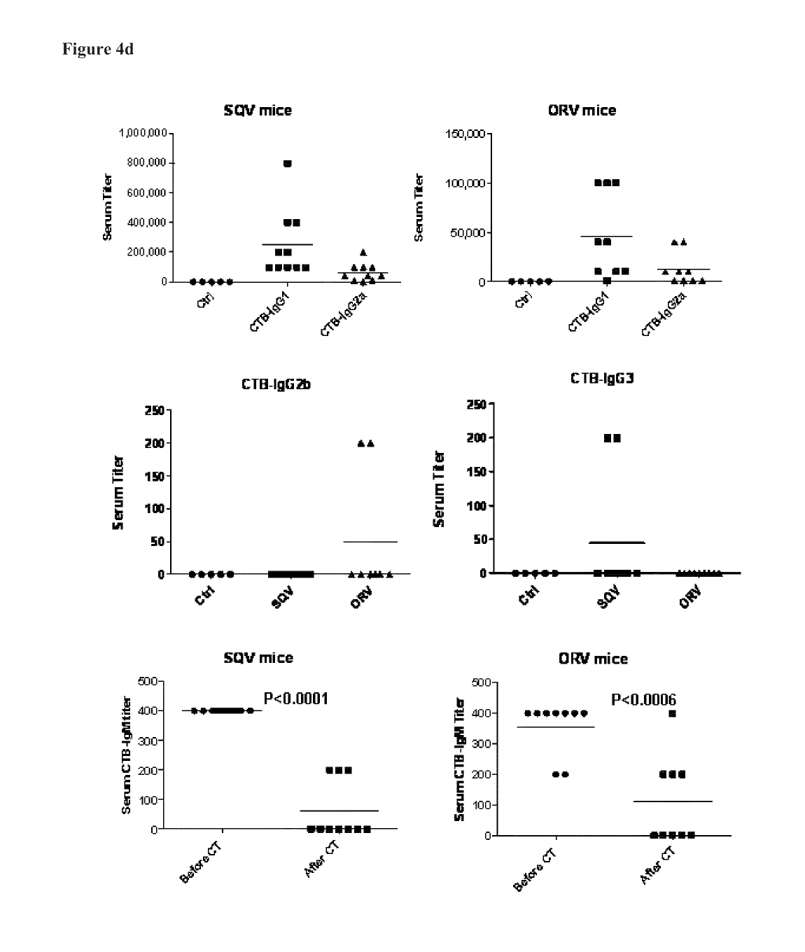

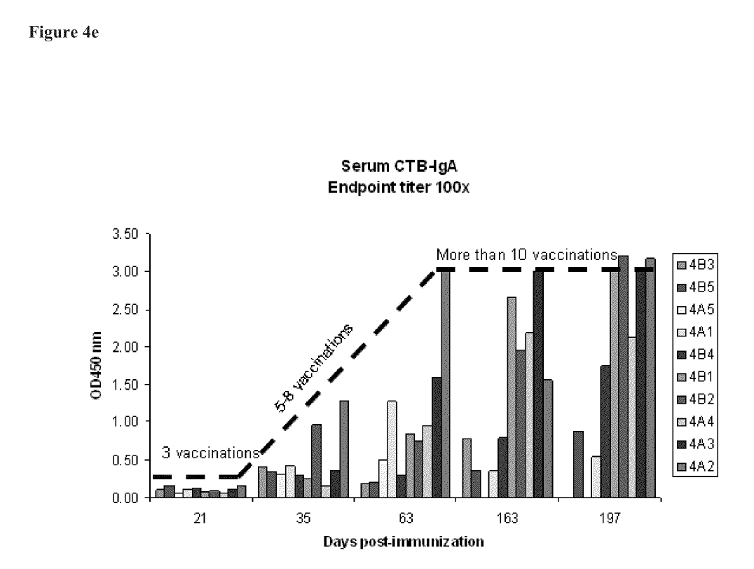

A broad range of CT concentration has been used by investigators (Guidry, J. J., Cardenas, L., Cheng, E. & Clements, J. D. Role of receptor binding in toxicity, immunogenicity, and adjuvanticity of Escherichia coli heat-labile enterotoxin. Infect. Immun. 65, 4943-4950 (1997); Chikwamba, R. et al. A functional antigen in a practical crop: LT-B producing maize protects mice against Escherichia coli heat labile enterotoxin (LT) and cholera toxin (CT). Transgenic Res. 11, 479-493 (2002); Bowman, C. C. & Clements, J. D. Differential biological and adjuvant activities of cholera toxin and Escherichia coli heat-labile enterotoxin hybrids. Infect. Immun. 69, 1528-1535 (2001); Glenn, G. M. et al. Transcutaneous immunization with cholera toxin protects mice against lethal mucosal toxin challenge. J. Immunol. 161, 3211-3214 (1998); Apter, F. M. et al. Analysis of the roles of antilipopolysaccharide and anti-cholera toxin immunoglobulin A (IgA) antibodies in protection against Vibrio cholerae and cholera toxin by use of monoclonal IgA antibodies in vivo. Infect. Immun. 61, 5279-5285 (1993)). BALB/c mice immunized with adjuvant (AJV), subcutaneous (SQV) or orally immunized with plant cells expressing CTB (ORV-CTB) or untransformed leaves (ORV-UT) were challenged with cholera toxin as described above. We found a significant association between the volume of intestinal water retention in SQV and ORV-CTB mice and subcutaneous or oral immunization with CTB (FIG. 4b). However, there was no significant difference in intestinal water content between SQV and ORV-CTB mice as shown in FIG. 4b. Control mice immunized with adjuvant (AJV) or gavaged with untransformed leaf developed severe diarrhea (FIG. 4a-d).

Our antigen-specific ELISA data showed that presence of serum and intestinal CTB-IgA in ORV-CTB mice but not in SQV, AJV and/or in control mice suggesting a direct correlation between IgA and protection in orally vaccinated mice (FIG. 4c). It should be noted that IgA titers repeatedly and reproducibly observed in ORV-CTB mice are much higher than those reported in previous studies. In contrast, in SQV mice that were protected from CT challenge, we were unable to detect any CTB-IgA in serum and/or in intestine by ELISA. To investigate the mechanism of protection observed in SQV mice, we screened a broad range of antigen-specific immunoglobulins by ELISA including -IgG1, -IgG2a, -IgG2b, -IgG3 and -IgM in the sera of vaccinated and control mice. Our data show that only CTB-IgG1 and no other tested immunoglobulin in this study conferred protection in SQV mice (FIG. 4d). Again, it should be noted that the mean IgG1 titer observed in SQV mice was about 250,000. Screening of the same profile of immunoglobulins in the sera of ORV mice showed comparable pattern of expression with SQV mice as shown in FIG. 4d, in addition to intestinal and serum IgA, suggesting that oral vaccination provides both mucosal and systemic immune response in contrast to subcutaneous immunization that provides only systemic immune response. Furthermore, we screened CTBIgG1, -IgG2a, -IgG2b, -IgG3 and -IgM in the sera of vaccinated mice before and after CTchallenged and our data show that only CTB -IgM level significantly changed after CT challenge (FIG. 4d).

The inventors also screened expression of IL-4 (Th2), IL-10 (Th2), IL-2 (Th1), IFNγ (Th1) and IL-17A (Th17) by ELISA in the sera of our experimental and control groups. Our data show that expression of IFNγ was detectable in 70% (7 of 10 mice), 16.6% (1 of 6 mouse) and 10% (1 of 10 mice) of control, SQV and ORV-CTB mice, respectively suggesting blocking of Th1 immune response in vaccinated mice. IL-17A is unlikely to play a role in this system because only one mouse in AJV and ORV-CTB groups were positive for this cytokine.

The inventors also determined minimum number of vaccination to generate adequate antigen specific antibody for effective protection from toxin challenge. As illustrated in FIG. 4e, it appears that a total number of 5 vaccinations are sufficient to reach to >90% immunity. Although subsequent boosters increased or decreased IgA titers in individual mice, all of them were protected from toxin challenge, despite 8-10 fold difference in IgA titers. This information is useful for generation of effective vaccination regiment with optimal number of boosters. Most of the currently used vaccines required 3-5 boosters (http://www.cdc.gov/).

E. Response of Cellular Components of the Immune System to CT Challenge

In order to study the impact of immunization on cellular components of the immune system, we measured expression of different markers associated with regulatory T-cells in fresh splenocytes obtained from controls (unvaccinated) and vaccinated mice after CT challenge. As shown in FIG. 5 (top row), CT challenge dramatically ameliorates numbers of CD4₊Foxp3₊ regulatory T-cell in unvaccinated control mice (increased from 11% to ˜25%). However, this effect was moderate in SQV (range from 7.2-12.5%) and ORV-CTB mice (range from 11.5-14%). As shown in FIG. 5, CT decreases expression of IL7Rα in unvaccinated mice but had marked upregulation in SQV and ORV-CTB mice. CT challenge eliminated CD4₊IL10₊ T-cells in unvaccinated control mice but significantly ameliorated this population in SQV and ORVCTB mice, for ˜12% and 7.5%, respectively (FIG. 5). To this end, CT upregulated expression of co-stimulatory signal CD80 in CD11c₊ splenic dendritic cells in unvaccinated control mice but this effect was neutral in vaccinated mice (FIG. 5).

F. Discussion

Production of an oral vaccine for cholera with ease of administration and that does not require cold chain is an important need, especially in areas with limited access to cold storage or transportation. Considering that mucosal surface is the site for many gastrointestinal, respiratory and urogenital infections, developing an oral vaccine has great significance. For instance, gastrointestinal infections caused by V. cholerae, Helicobacter pylori, Shigella spp and/or by rotaviruses, Entamoeba histolytica are major examples among many others.

The investigation described herein is the longest cholera vaccine study reported so for in the plant vaccine literature. Animals were boosted until 267 days and were challenged on day 303. Therefore, this study provides documentation on the longevity of mucosal and systemic immunity. This observation is significant in the light of recent reports on waning immunity against cholera King, A. A., Ionides, E. L., Pascual, M. & Bouma, M. J. Inapparent infections and cholera dynamics. Nature 454, 877-880 (2008)). With the current cholera vaccine, immunity is lost in children within three years and adults are not fully protected Olsson, L. & Parment, P. A. Present and future cholera vaccines. Expert. Rev. Vaccines 5, 751-752 (2006). Although boosters beyond 5-8 did not significantly increase immunity levels, long-term protection was maintained. Considering the life span of BALB/c (˜2 years), this translates into protection up to 50% of mouse life span. Another interesting aspect of our study is the analysis of immunoglobulin in individual mice in each group whereas most previously reported studies used pooled sera for each group. Even though BALB/c mice are inbred strains, 8-10 fold variability observed within each group sheds new light on the correlation between immune titers and conferred protection. Such data should be valuable in prediction of protection in human clinical studies, amidst such variable immune response. The highest level of immune titers reported in this study may be due to high levels of CTB expression in chloroplasts and not larger number of boosters given because most previous studies have given up to 6 or 8 oral boosters or the same number of subcutaneous boosters as used in our study. In the current study, we observed high level of CTB-IgA only in ORV-CTB mice but not in SQV or AJV or ORV-UT mice. In contrast, antigen presentation to the mucosal immune system via a non-receptor mediated delivery resulted in little or no local antigen-specific IgA (Arlen, P. A. et al. Effective plague vaccination via oral delivery of plant cells expressing F1-V antigens in chloroplasts. Infect. Immun. 76, 3640-3650 (2008)). These data suggest that induction of intestinal IgA may require a receptor-mediated antigen presentation to the gut immune system and the antigen should be presented to the gut mucosal immune system and not to any other part of the systemic immune system. Further studies with antigens conjugated with and without CTB or other proteins that bind to intestinal receptors are necessary to understand the relationship between antigen presentation and production of IgA.

Recently it has been shown that interaction of intestinal IgA with other locally generated cytokines such as TGFβ1, IL-10 and IL-4 will provide a unique microenvironment to educate DCs and subsequently educated DCs will imprint naïve T-cells₃₈ and imprinted T-cells secretes the same cytokine profile as previously antigen-experienced T-cells. None of SQV mice had detectable CTB-IgA; however, 89% of SQV mice were protected from CT challenge. Our data show that only serum CTB-IgG1 and not -IgG2a, -IgG2b, -IgG3 or -IgM confers immunity against CT challenge in SQV mice. Our data show that only CTB-IgM significantly decreased after CT challenge, while other members of the family remained the same. Our study has evaluated more immunoglobulins in response to delivery of plant-derived vaccine antigens than previous studies but further studies are needed to understand this process. Furthermore, our data from single-cell based studies suggest that CT increased numbers of Foxp3₊ regulatory T-cells and co-stimulatory molecule CD80 in splenocytes in unvaccinated control mice but CT had little effect on this population in vaccinated mice. Increasing numbers of Foxp3 regulatory T-cells in unvaccinated mice is interesting because this population is the most effective arm of peripheral tolerance. Immediate consequences of higher numbers of Foxp3₊ regulatory T-cell would be suppression of responding T-cell populations to CT (Shevach, E. M. CD4+CD25+ suppressor T cells: more questions than answers. Nat. Rev. Immunol. 2, 389-400 (2002)).

Because CT did not increase numbers of CD4₊CD25_{+high} T-cells (data not shown), it appears that CT converts Foxp3−CD25−CD4₊ T-cells into Foxp3₊ regulatory T-cells in the periphery. In agreement with our data, recently Sun et al. have reported increasing number of Ag-specific Foxp3₊ regulatory T-cells by CTB and CTB plus CT, respectively (Sun, J. B., Raghavan, S., Sjoling, A., Lundin, S & Holmgren, J. Oral tolerance induction with antigen conjugated to cholera toxin B subunit generates both Foxp3+CD25+ and Foxp3−CD25−CD4+ regulatory T cells. J. Immunol. 177, 7634-7644 (2006)). They have also demonstrated that intragasteric administration of OVA-CTB induced expansion of antigen-specific Foxp3₊CD25₊ regulatory T-cells, when compared with the sham treated control mice. In our study, CT induced upregulation of IL-10 expressing CD4₊ T-cells, CTB-IgA and CTBIgG1 in ORV and SQV, respectively, suggesting that vaccination regiment induced a Tr1/Th2 immune response and protected vaccinated mice against CT challenge. Upregulation of IL-7Rα+Foxp3₋CD4₊ T-cell in vaccinated mice after CT challenge is interesting because it has been reported that formation of Peyer's patches is dependent upon IL-7 receptor, TNF and TNF superfamily members (Fu, Y. X. & Chaplin, D. D. Development and maturation of secondary lymphoid tissues. Annu. Rev. Immunol. 17, 399-433 (1999)). Further experiments are needed to address functional properties of IL-7R in plant-derived vaccines and immunity. In conclusion, this study demonstrates efficacy of an inexpensive vaccination method using transgenic plant-derived leaf to protect mice from CT challenge. Currently, other than the polio vaccine and the rotavirus, there are no other examples of oral vaccines in the US and the mucosal immune system has not been utilized to confer immunity against invading pathogens. Oral polio vaccine was discontinued in the US because one in 2.4 million cases contracted polio from the live attenuated oral vaccine. However, such problems are not associated with subunit vaccines because only one or two antigens are used that are incapable of causing any disease.

Therefore, it is important to understand and utilize the mucosal immune system for delivery of subunit vaccines. Bioencapsulation of vaccine antigens in plant cells provide an ideal low cost delivery system for large-scale distribution at times of crisis. It is important to point out that oral delivery confers dual protection via systemic and mucosal immune systems. High level and long-term protection observed against cholera toxin challenge using chloroplast-derived antigen, makes this system yet another new platform for advancing towards human clinical studies.

G. Methods

G.1 Chloroplast Vector Construction and Regeneration of Transplastomic Plants.

The pUC based Lactuca sativa long flanking plasmid sequence (pLSLF)₂₄ was used to integrate foreign genes into the intergenic spacer region between the trnI (Ile) and trnA (Ala) genes as described previously Ruhlman, T., Ahangari, R., Devine, A., Samsam, M. & Daniell, H. Expression of cholera toxin B-proinsulin fusion protein in lettuce and tobacco chloroplasts—oral administration protects against development of insulitis in non-obese diabetic mice. Plant Biotechnol. J 5, 495-510 (2007); Verma, D., Samson, N. P., Koya, V. & Daniell, H. A protocol for expression of foreign genes in chloroplasts. Nat. Protoc. 3, 739-758 (2008)). The lettuce native 16s ribosomal operon promoter, and 3′ rbcL were amplified from the lettuce chloroplast genome. The CTB sequence was amplified using pLD-5′UTR-CTB-Pins₂₄ vector as the template. The final CTB expression cassette with the tobacco psbA promoter including 5′ untranslated regions (UTR) and the tobacco psbA 3′ UTR was cloned into pLsDV vector resulting in the lettuce chloroplast vector pLsDV CTB. Lactuca sativa var. Simpson elite was transformed and the transplastomic lines were selected as described previously Kanamoto, H. et al. Efficient and stable transformation of Lactuca sativa L. cv. Cisco (lettuce) plastids. Transgenic Res. 15, 205-217 (2006)). Shoots were screened by PCR for the confirmation of transplastomic lines and PCR positive shoots were subjected to additional rounds of selection and regeneration. Rooted transplastomic lines were hardened in Jiffy® peat pots before transfer to the green house.

G.2 Confirmation of Transgene Integration and Expression

PCR reactions were performed using two sets of primers namely 16SF/3M and 5P/2M. Southern blot analysis was performed to confirm transgene integration as well as homoplasmy as described earlier (Kumar, S. & Daniell, H. Engineering the chloroplast genome for hyperexpression of human therapeutic proteins and vaccine antigens. Methods Mol. Biol. 267, 365-383 (2004)). Total plant DNA (1-2 μg) isolated from control and transplastomic lines was digested with SmaI and probed with lettuce flanking sequence DNA. Chloroplast vector pLsDV CTB was digested with NdeI and XbaI to generate a 0.322 kb CTB probe. After labeling the probes with ₃₂Pα[dCTP], the membranes were hybridized by using Stratagene Quik-Hyb® hybridization solution following the manufacturer protocol (Stratagene, La Jolla, Calif.). Approximately 100 mg of leaf was ground in liquid nitrogen and used for western blot analysis as described previously (Kumar and Daniell 2004).

G.3 Mice and Immunization Schedule

Female BALB/c mice (Jackson Laboratories) were housed at the University of Central Florida mouse facility in ventilated cages under specific pathogen-free (SPF) conditions. All mice and procedures performed in this study are based on an approved protocol and are in accordance with the UCF-IACUC. Ten week old mice were randomly divided into control oral gavage with untransformed leaf (n=5), adjuvant (n=5), subcutaneous (n=9) and oral transplastomic group (n=10). Chloroplast-derived CTB fusion proteins were bound to adjuvant and injected into the scruff of the neck (25 μg) and 500 mg of transgenic plant materials was orally gavaged using an insulin syringe equipped with a 27-gauge stainless steel ball-ended needle as described elsewhere (Schreiber, M. Evaluation of the efficacy of chloroplast-derived antigens against malaria. Master's thesis, College of Medicine, University of Central Florida, Orlando, Fla. (2008)).

Mice in subcutaneous group received six boosts on days 13, 27, 43, 55, 155 and 129. Mice in oral gavage group received boosts of on days 0, 10, 17, 24, 31, 37, 45, 52, 59, 150, 157, 189 and 219.

G.4 CHO Elongation Assay

CHO cell elongation assays were performed as described 12 with suitable modifications. In brief, CHO cells were seeded in 96-flat well plates (50,000 cells/well) and incubated at 37° C. for 12-16 hr. A 3-fold dilution of pooled sera (5 mice) from different groups of mice were neutralized with CT (50 ng/ml) at 37° C. for 1 hr and 100 μl of neutralized sera was replaced with 50 μl cell culture supernatant and incubation was continued at 37° C. for 12 hr. Cell viability was examined with trypan blue exclusion method.

G.5 GM1 Binding ELISA Assays

Functionality of chloroplast derived CTB was checked by CTB-GM1 binding assay. Ninety six-well plates were coated with 100 μl of monosialoganglioside-GM₁ (3.0 ng/ml in bicarbonate buffer) and non fat milk as a control, and then incubated overnight at 4° C. Primary and secondary antibodies were used at dilutions similar to those in the western blot protocol. Following washing, 100 μl of 3,3,5,5-tetramethylbenzidine (TMB, American Qualex) was added to each well and incubated in the dark for 20 min. The reaction was stopped by adding 50 μl 2N H₂SO₄ and plate was read on a microplate reader (BIORAD) at 450 nm.

G.6 Determination of CT Dose for In Vivo Challenge

Cholera toxin (CT, Sigma, C8052) was diluted (final concentration 1 mg/ml) in PBS buffer containing 6% NaHCO3 and 0.5% albumin. Five unvaccinated BALB/c mice with the same age and sex as of our experimental group were given different doses of CT (1, 1.5, 2, 3 and 4 μg/g of body weight) for 14 hr. The mice remained in their cages without food but water ad labitum. The mice were scarified after 14 hr and intestinal water retention was collected and measured.

G.7 ELISA

Sandwich ELISA was performed on transgenic lettuce leaf materials expressing CTB and mice sera for cytokines detection. In brief, the standards and transgenic samples were diluted in coating buffer and coated on a 96-well plate overnight at 4° C. The remainder of the procedure was similar to GM₁ binding assay described above. Sandwich ELISA for different cytokines was performed as described. Plates (96-well) were coated with anti-mouse IL-2 (2 μg/ml), IL-4 (2 μg/ml), IFNγ (2 μg/ml), IL-17A (2 μg/ml) antibodies (all from eBioscinces) using carbonate buffer (pH=9.6) at 4C for 12-16 hr. Plates were washed and hybridized with diluted sera at 37C for 1 hr. Detection was performed as described earlier. Capture ELISA for CTB antigen-specific IgA, IgG1, IgG2a, IgG2b, IgG3, IgM antibodies in sera and intestinal content (IgA only) of different group of mice were performed by coating 96-well flat bottom plate with 1 μg/ml (100 μl) of CTB (Sigma) in carbonate buffer (pH=9.7) at 4C for 12-16 hr. Plates were washed, blocked and hybridized with diluted sera with biotin-conjugated antibodies as follows: rat anti-mouse IgG1, IgG2a, IgG2b, IgG3 and IgM (all from Southern Biotech, AL). HRP-conjugated streptavidin (Peirce, 1:4000) and TMB were used for detection and substrate, respectively. For CTB antigen specific IgA in sera and intestinal content, goat-anti mouse IgA-HRP (American Qualex, 1: 2000) was used. Rabbit anti-CTB Ab (1:4000, Sigma) and anti-rabbit IgG-HRP Ab (1:7500) were used as primary and secondary antibodies for CTB, respectively.

G.8 Flow Cytometry

Flow cytometry analysis was performed on fresh single-cell suspension of splenocytes. Cell surface staining on freshly prepared splenocytes was performed using anti-mouse CD4 (L3T4, BD Pharmingen), CD25 (3C7, BD Pharmingen), CD127 (IL-7Ra)(SB/199, BD Pharmingen), CD44 (IM7, BD Pharmingen), CD11c (HL3, BD Pharmingen), CD80 (16-10A1, eBioscience), biotin-conjugated MHC II (M5/114.15.2, eBioscience). Purified rat anti-mouse CD16/CD32 (2.4G2, BD Pharmingen) was used to block Fc receptor in myeloid cell lineages. Intra-cellular staining of Foxp3 (FJK, 16S, eBioscience), IL-4 (11B11, eBiosciences), IL-10 (JES5-16E3, eBioscience), IFNγ (XMG1.2, eBioscience) was performed using foxp3 intra cellular staining kit (eBioscience) according to instructions provided by manufacturer. Flow cytometry was performed using FACSCalibur (BD Bioscience) and 30,000 events were acquired for each condition and data analysis was performed using FCS express (v3) software (De Novo soft ware).

G.9 Statistical Analysis

Data are reported as the mean±SD. All analyses for statistically significant differences were performed using One-way ANOVA and the t test (GraphPad Prism 5) andp values less than 0.05% considered significant.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein. U.S. Patent Publication 20030009783 and 20060031964 are also cited for plant transformation techniques.

### Example 2

### Dual Chloroplast Derived Oral and Injectable Vaccines Against Cholera and Malaria

A. Materials and Methods

A.1 Chloroplast Vector Construction

The pUC based Lactuca sativa long flanking plasmid sequence (pLSLF) (Ruhlman T, Ahangari R, Devine A, Samsam M, Daniell H (2007) Expression of cholera toxin B-proinsulin fusion protein in lettuce and tobacco chloroplasts—oral administration protects against development of insulitis in non-obese diabetic mice. Plant Biotechnol J 5:495-510) was used to integrate foreign genes into the intergenic spacer region between the trnI (Ile) and trnA (Ala) genes as described previously (Ruhlman et al. 2007). Chloroplast transformation vectors were constructed as previously described, using standard molecular biology protocols (Verma D, Samson N P, Koya V, Daniell H (2008) A protocol for expression of foreign genes in chloroplasts. Nat Protoc 3:739-758). The Prrn:aadA:rbcL selectable marker gene cassette contained rrn promoter and rbcL 3′ untranslated region (UTR) amplified from the lettuce chloroplast genome. The CTB sequence was amplified using pLD-5′UTR-CTB-Pins (Ruhlman et al. 2007) vector as the template. The final CTB expression cassette with the tobacco psbA promoter including 5′ untranslated regions (UTR) and the tobacco psbA 3′ UTR was cloned into pLsDV vector resulting in the lettuce chloroplast vector pLsDV CTB. The AMA1 and MSP1 were synthesized according to Pan et al. (Pan W, Huang D, Zhang Q, Qu L, Zhang D et al. (2004) Fusion of two malaria vaccine candidate antigens enhances product yield, immunogenicity, and antibody-mediated inhibition of parasite growth in vitro. J Immunol 172:6167-6174), and cloned into the pGEMT Easy Vector (Promega) and the sequences were confirmed and subcloned into the pBSK+ (Stratagene) vector. The pLsDV CTB-AMA1 and pLsDV CTB-MSP1 was constructed using endogenous psbA promoter, 5′ UTR and 3′ UTR from lettuce. CTB-AMA1 fusion had GPGP hinge region and the furin cleavage site while CTB-MSP1 only had the GPGP hinge in between fusion proteins to facilitate correct folding of each protein by reducing the steric hindrance.

A.2 Regeneration of Transplastomic Plants

Leaves of Nicotiana tabacum var. Petite Havana were bombarded with pLD CTB-FC-AMA1 and pLD CTB-MSP1 and the transformants were obtained as described (Kumar S, Daniell H (2004) Engineering the chloroplast genome for hyperexpression of human therapeutic proteins and vaccine antigens. Methods Mol Biol 267:365-383). Leaves of Lactuca sativa var. Simpson elite were bombarded with pLsDV CTB, pLsDV CTB-AMA1 and pLsDV CTB-MSP1 and the transplastomic lines were selected as described previously (Ruhlman et al. 2007; Kanamoto H, Yamashita A, Asao H, Okumura S, Takase H et al. (2006) Efficient and stable transformation of Lactuca sativa L. cv. Cisco (lettuce) plastids. Transgenic Res 15:205-217). Shoots were screened by PCR for the confirmation of transplastomic lines and PCR positive shoots were subjected to additional rounds of selection and regeneration. Rooted transplastomic lines were hardened in Jiffy® peat pots before transfer to the green house.

A.3 Confirmation of Transgene Integration and Expression

PCR reactions were performed using two sets of primers namely 16SF/3M or 3P/3M and 5P/2M. Southern blot analysis was performed to confirm transgene integration as well as homoplasmy as described earlier. Total plant DNA (1-2 μg) isolated from control and transplastomic lines was digested with SmaI or HindIII for lettuce, ApaI for tobacco and probed with 1.13 kb of lettuce flanking sequence DNA or 0.8 kb of tobacco flanking sequence, respectively. Chloroplast vector pLsDV CTB was digested with NdeI and XbaI to generate a 0.322 kb CTB probe. After labeling the probes with ³²Pα[dCTP], the membranes were hybridized by using Stratagene Quik-Hyb® hybridization solution following the manufacturer protocol (Stratagene, La Jolla, Calif.).

A.4 Immunoblot Analysis

After estimation of total soluble protein (TSP) using Bradford method, 10 μg of TSP from sample was separated in 12% sodium dodecylsulphate-polyacrylamide gel electrophoresis (SDS-PAGE) and transferred to nitrocellulose membranes for immunoblotting, according to Verma et al. 2008. The protein separated by SDS-PAGE gel was transferred to nitrocellulose membrane by electroblotting and the membrane was blocked overnight with 3% non-fat dry milk. To detect CTB, CTB-FC-AMA1 and CTB-MSP1 fused proteins, blots were incubated with 1:3000 rabbit anti-CTB primary polyclonal antibody (Sigma, St. Louis, Mo., USA) followed by 1:5000 HRP-conjugated donkey anti-rabbit secondary antibody (Southernbiotech, Birmingham, Ala., USA). A SuperSignal® West Pico chemiluminescence substrate Kit (Pierce, Rockford, Ill., USA) was used for autoradiographic detection.

A.5 Enrichment of Chloroplast-Derived Proteins

Chloroplast-derived CTB-malarial proteins were extracted by grinding of lOg freeze dried leaf materials in 20 ml of plant extraction buffer (100 mM NaCl, 200 mM Tris-HCl pH8, 0.05% Tween 20, 0.1% SDS, 200 mM sucrose, containing the Roche complete mini EDTA-free protease inhibitor cocktail). The samples were placed on ice and homogenized for five minutes with an OMNI International (GLH-2596) probe and centrifuged at 14,000 rpm for 15 minutes at 4° C. The supernatant was collected and then subjected to TALON superflow Metal Affinity Resin (Clontech) to enrich the chloroplast-derived CTB-malarial proteins according to manufacturer's instructions. The eluted fraction along with other fractions such as washes and flow through was collected and subjected to the Bradford Protein assay (BioRad) and to the RC-DC Protein Assay (Bio-Rad) to determine protein concentration. The eluted fractions were dialyzed with sterile PBS and the Slide-A-Lyzer Dialysis Cassette 10,000 MW (PIERCE).

A.6 Conjugation of Chloroplast-Derived Protein to Adjuvant

Chloroplast-enriched proteins (˜2.5 mg) were mixed with 1:4 diluted alhydrogel in PBS (Aluminum Hydroxide Gel, Sigma) and incubated overnight with gentle rocking at 4° C. The samples were centrifuged at 2,000 x g for five minutes at 4° C. The RC-DC Protein Assay (Bio-Rad) was used to determine efficiency of conjugation by comparing the total amount of protein added to the adjuvant and the protein remaining in the supernatant after binding to adjuvant. The conjugated protein pellet was resuspended in sterile PBS to a final concentration of 1 μg/μl.

A.7 Mice Immunization Schedule and CT Challenge

Ninety female BALB/c mice, purchased from the Charles River Laboratories at 7 week of age, were housed at the University of Central Florida mouse facility in ventilated cages under specific pathogen-free (SPF) conditions. All mice and procedures performed in this study are based on an approved protocol and are in accordance with the UCF-IACUC. Mice were randomly divided into nine groups (n=10 per group): group 1: oral UT group gavaged with untransformed leaves; group 2: adjuvant with no bound antigen; group 3: CTB-AMA1 purified antigen with adjuvant; group 4: oral gavage with leaves expressing CTB-AMA1; group 5: CTB-MSP1 purified antigen with adjuvant; group 6: oral gavage with leaves expressing CTB-MSP1; group 7, CTB-MSP1 & CTB-AMA1 purified antigens bound with adjuvant; group 8: oral gavage with leaves expressing CTB-AMA1 & MSP1 and group 9: untreated mice. Mice in groups 2-8 were initially primed subcutaneously with corresponding antigen followed by oral and/or subcutaneous boosts in the course of this study.

Chloroplast-derived CTB fusion proteins were bound to adjuvant and injected into the scruff of the neck (25 μg) and 500 mg of transgenic plant materials was orally gavaged using an insulin syringe equipped with a 27-gauge stainless steel ball-ended needle as described elsewhere (Schreiber M (2008) Evaluation of the efficacy of chloroplast-derived antigens against malaria. Master's thesis, College of Medicine, University of Central Fla., Orlando, Fla.). Mice in subcutaneous group received six boosts on days 13, 27, 43, 55, 155 and 129. Mice in oral gavage group received boosts on days 10, 17, 24, 31, 37, 45, 52, 59, 150, 157, 189 and 219. Effective dose of CT was empirically determined in experimental/control mice cohort (data not shown). Mice were orally challenged with 100 μl of CT (Sigma, 30 μg/mouse) for 14 hr and mice had unlimited access to water but not food. Mice were then euthanized and intestinal content was collected and various organs of the immune system were harvested for further analysis.

A.8 CHO Elongation Assay

CHO cell elongation assays were performed as described [34] with suitable modifications. In brief, CHO cells were seeded in 96-flat well plates (50,000 cells/well) and incubated at 37° C. for 12-16 hr. A 3-fold dilution of pooled sera (5 mice) from different groups of mice were neutralized with CT (50 ng/ml) at 37° C. for 1 hr and 100 μl of neutralized sera was replaced with 50 μl cell culture supernatant and incubation was continued at 37° C. for 12 hr. Cell viability was examined with trypan blue exclusion method.

A.9 GM1 Binding ELISA Assays

Functionality of chloroplast derived CTB was checked by CTB-GM₁ binding assay. Ninety six-well plates were coated with 100 μl of monosialoganglioside-GM₁ (3.0 ng/ml in bicarbonate buffer) and non fat milk as a control, and then incubated overnight at 4° C. Primary and secondary antibodies were used at dilutions similar to those in the western blot protocol. Following washing, 100 μl of 3,3,5,5-tetramethylbenzidine (TMB, American Qualex) was added to each well and incubated in the dark for 20 min. The reaction was stopped by adding 50 μl 2N H₂SO₄ and plate was read on a microplate reader (BIORAD) at 450 nm.

A.10 ELISA

Sandwich ELISA was performed on transgenic lettuce leaf materials expressing CTB and mice sera for cytokines detection. In brief, the standards and transgenic samples were diluted in coating buffer and coated on a 96-well plate overnight at 4° C. The remainder of the procedure was similar to GM₁ binding assay described above.

Sandwich ELISA for different cytokines was performed as described (Arlen P A, Singleton M, Adamovicz J J, Ding Y, Davoodi-Semiromi A et al. (2008) Effective plague vaccination via oral delivery of plant cells expressing F1-V antigens in chloroplasts. Infect Immun 76:3640-3650). Plates (flat bottom 96-well) were coated with anti-mouse IL-2 (2 μg/ml), IL-4 (2 μg/ml), IFNγ (2 μg/ml), IL-17A (2 μg/ml) (all from eBioscinces) using carbonate buffer (pH=9.6) at 4° C. for 12-16 hr. Plates were washed and hybridized with diluted sera at 37° C. for 1 hr. Detection was performed as described earlier.

Capture ELISA for MSP1(MSP1 polypeptide was obtained from the Malaria research and Reference Reagent Resource, MR, managed by ATCC, Manassas, Va.) and CTB antigen-specific IgA, IgG1, IgG2a, IgG2b, IgG3, IgM antibodies in sera and intestinal content (IgA only) of different group of mice were performed by coating 96-well flat bottom plate with 1 μg/ml (100 μl) of CTB (Sigma) or MSP1 polypeptide in carbonate buffer (pH=9.7) at 4° C. for 12-16 hr. Plates were washed, blocked and hybridized with diluted sera with biotin-conjugated antibodies as follows: rat anti-mouse IgG1, IgG2a, IgG2b, IgG3 and IgM (all from Southern Biotech, AL). HRP-conjugated streptavidin (Peirce, 1:4,000) and TMB were used for detection and substrate, respectively. For CTB antigen specific IgA in sera and intestinal content, goat-anti mouse IgA-HRP (American Qualex, 1:2,000) was used. Rabbit anti-CTB Ab (1:4,000, Sigma) and anti-rabbit IgG-HRP Ab (1:7,500) were used as primary and secondary antibodies for CTB, respectively.

A.11 Flow Cytometry

Flow cytometry analysis was performed on fresh single-cell suspension of splenocytes. Cell surface staining on freshly prepared splenocytes was performed using anti-mouse CD4 (L3T4, BD Pharmingen), CD25 (3C7, BD Pharmingen), CD127 (IL-7Ra)(SB/199, BD Pharmingen), CD44 (IM7, BD Pharmingen), CD11c (HL3, BD Pharmingen), CD80 (16-10A1, eBioscience), biotin-conjugated MHC II (M5/114.15.2, eBioscience). Purified rat anti-mouse CD16/CD32 (2.4G2, BD Pharmingen) was used to block Fc receptor in myeloid cell lineages.

Intra-cellular staining of Foxp3 (FJK, 16S, eBioscience), IL-4 (11B11, eBiosciences), IL-10 (JES5-16E3, eBioscience), IFNγ (XMG1.2, eBioscience) was performed using foxp3 intra cellular staining kit (eBioscience) according to instructions provided by manufacturer. Flow cytometry was performed using FACSCalibur (BD Bioscience) and 30,000 events were acquired for each condition and data analysis was performed using FCS express (v3) software (De Novo software).

A.12 Immunofluorescence Detection of Malarial Antigens with Sera of Vaccinated Mice

A revised protocol described by Tonkin et al. was adopted for the preparation and fixation of RBCs (Tonkin C J, van Dooren G G, Spurck T P, Struck N S, Good R T et al. (2004) Localization of organellar proteins in Plasmodium falciparum using a novel set of transfection vectors and a new immunofluorescence fixation method. Mol Biochem Parasitol 137:13-21) and by Ayong et al. for detection of antigen with immunofluorescence (Ayong L, Pagnotti G, Tobon A B, Chakrabarti D (2007) Identification of Plasmodium falciparum family of SNAREs. Mol Biochem Parasitol 152:113-122). Diluted sera (1:500) were hybridized on RBCs followed by hybridization with diluted (1:1,000) Alexa Fluor 555 goat anti-mouse antibody. Cells were allowed to settle on previously coated coverslips with 1% PEI for thirty minutes at room temperature. The mounting solution, 50% glycerol with 0.1 mg/mL DABCO (Sigma) was added to cover slips and then inverted on microscope slides. Fluorescence images were observed and captured by the LSM 510 confocal laser scanning microscope (Carl Zeiss).

A.13 In Vitro Parasite Inhibition Assay

The 3D7 P. falciparum culture was synchronized with ring stage parasites with sorbitol lysis. The parasite completed one cycle and was allowed to mature to the trophozoite-schizont stage. The hematocrit and parasitemia were adjusted to 2% (2.5% parasitemia for the MRA-35 PfMSP1₁₉ in vitro parasite inhibition assay). Mouse sera and MRA-35 PfMSP1₁₉ (positive control) were heat inactivated at 56° C. for 30 minutes and hybridized on human RBCs overnight at 4° C. (Sachdeva S, Mohmmed A, Dasaradhi P V, Crabb B S, Katyal A et al. (2006) Immunogenicity and protective efficacy of Escherichia coli expressed Plasmodium falciparum merozoite surface protein-1(42) using human compatible adjuvants. Vaccine 24:2007-2016). The mouse serum was added to the parasite culture in 96-well plates at a final concentration of 20% (for the MRA-35 PfMSP1₁₉ in vitro parasite inhibition assay 5 μl of antibody was added and diluted 1:5-1:625 to 25 μl of parasite culture). To serve as a negative control, no serum was added to wells and replaced with culture media. The cultures were incubated for 48 hours to allow for schizont rupture and merozoite invasion. The assays were preformed in duplicate and repeated at least three times.

For microscopic analysis using the 100× oil immersion lens, blood smears were made and stained with Giemsa and the numbers of parasites per 900-1,100 RBCs were determined for each well. Parasitemia was measured using the following formula (infected RBCs/infected+uninfected RBCs)×100. Percent of inhibition was determined by the following formula (% parasitemia of no sera added−% parasitemia of experimental mouse sera/% parasitemia of no sera added)×100. Relative percent of inhibition was determined by the following formula (% of inhibition from experimental mouse sera/% inhibition of MRA-35 PfMSP1₁₉ (positive control))×100 and the percent of inhibition for the positive control was set at 100%.

A. 14 Statistical Analysis

Data are reported as the mean±SD. All analyses for statistically significant differences were performed using One-way ANOVA and the t test (GraphPad Prism 5) andp values less than 0.05% considered significant.

B. Results

B.1 Characterization of Lettuce and Tobacco Transplastomic Lines Expressing Vaccine Antigens

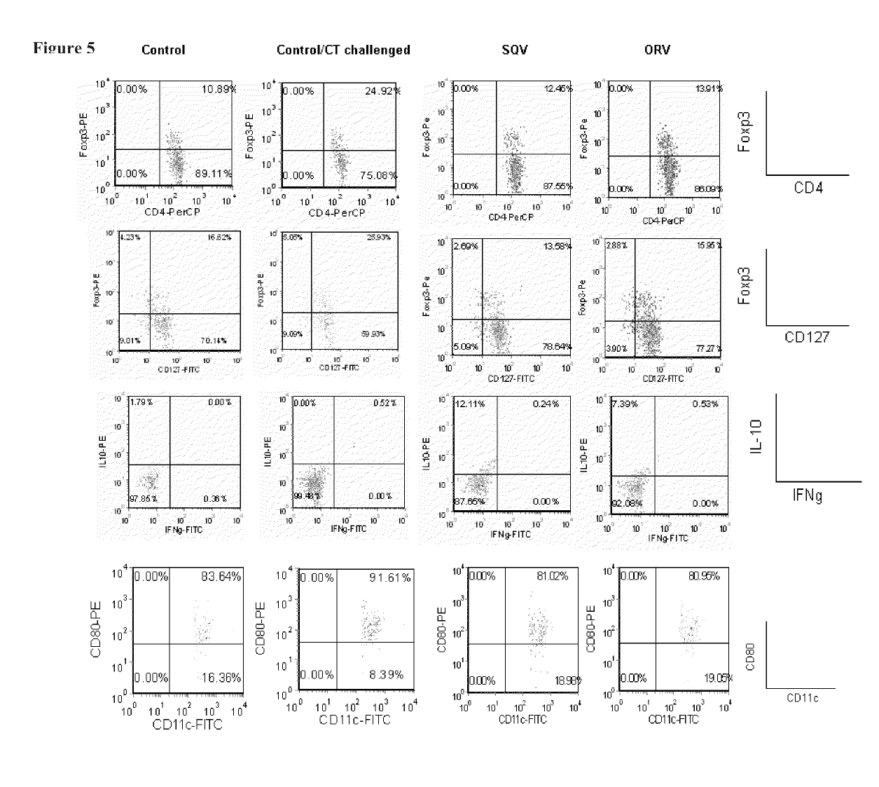

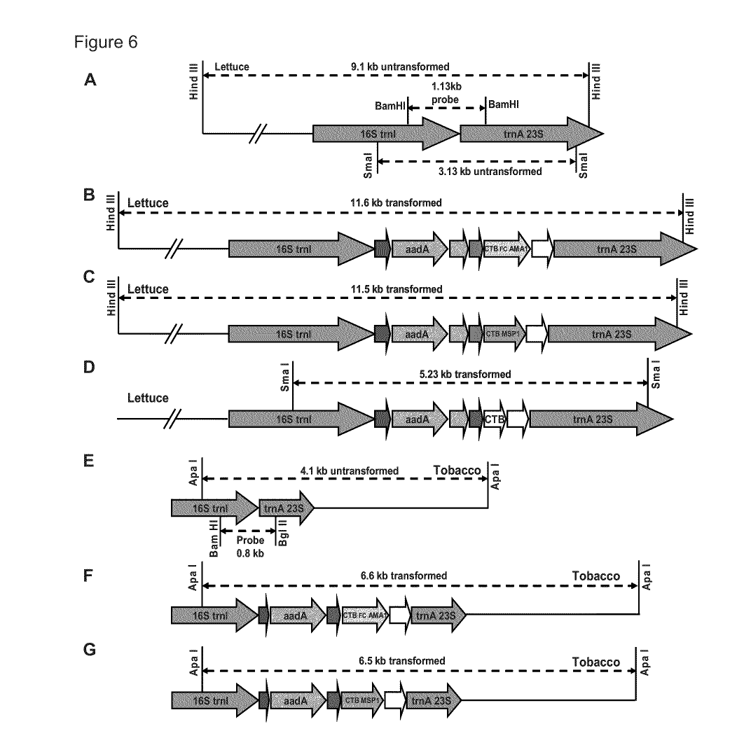

Tobacco chloroplast vectors contained the trnI (Ile) and trnA (Ala) genes for homologous recombination and expression cassettes for vaccine antigens CTB-AMA1 and CTB-MSP1 were regulated by the tobacco psbA promoter, 5′untranslated region to enhance translation and the 3′ untranslated region to confer transcript stability (FIG. 6). In lettuce chloroplast vectors, CTB expression cassette was regulated by the tobacco psbA promoter including 5′ untranslated regions (UTR) and the tobacco psbA 3′ UTR (FIG. 6). In this construct, 16S/trnI and trnA/23S genes were used as flanking sequences (longer flanking sequence than tobacco) for homologous recombination with the native chloroplast genome. Expression cassettes for vaccine antigens CTB-AMA1 and CTB-MSP1 in lettuce were regulated by endogenous psbA promoter, 5′untranslated region to enhance translation and the 3′ untranslated region to confer transcript stability (FIG. 6).

Transplastomic tobacco and lettuce plants were obtained as described previously (Ruhlman 2007). Five to six primary tobacco and 3-6 lettuce transformants appeared 3-6 weeks after bombardment from leaves placed on the regeneration medium containing the selection agent. Primary transformants were screened by PCR using 3P/3M and 5P/2M primer pairs in tobacco and 16SF/3M and 5P/2M primer pairs in lettuce (data not shown). Following an additional round of selective regeneration, progenitors for each transplastomic line was rooted in medium containing the selection agent. Clones were transferred to Jiffy0 peat pots, acclimatized in biodome and moved to the greenhouse, where they matured, flowered and produced seeds.

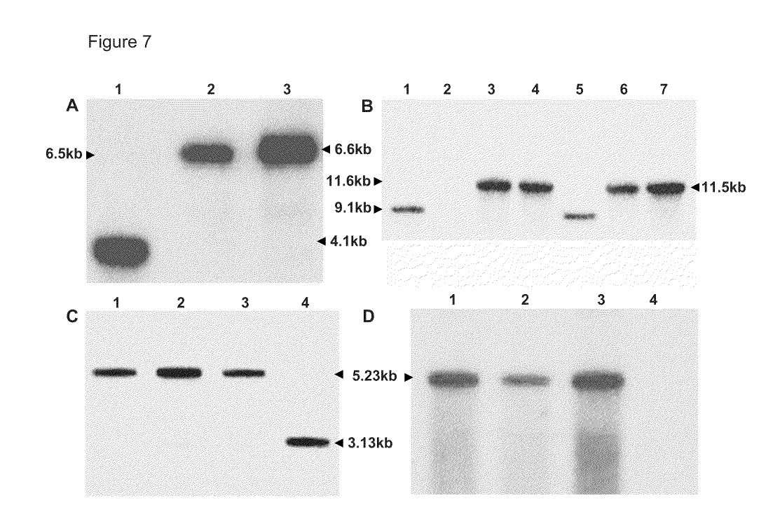

The site specific transgene integration into the chloroplast genome and homoplasmy were evaluated by Southern blot analysis in all tobacco and lettuce transgenic lines (FIG. 7). In tobacco, transplastomic lines with CTB-MSP1 yielded (6.5 kb), and with CTB-AMA1 yielded 6.6 kb fragments (FIG. 7A), while untransformed line yielded a 4.1 kb fragment (FIG. 7A). Lettuce transplastomic lines with CTB-AMA1 yielded 11.6 kb and CTB-MSP1 yielded 11.5 kb, while untransformed lines yielded 9.1 kb fragment (FIG. 7B). Lettuce transplastomic lines with CTB alone yielded a 5.23 kb fragment (FIG. 7C) and untransformed line yielded 3.13 kb fragment. The absence of untransformed fragment in lettuce and tobacco transplastomic lines confirms that they achieved homoplasmy. Presence of CTB in transplastomes was confirmed by the CTB probe (FIG. 7D).

B.3 Expression and Quantitation of Vaccine Antigens in Lettuce and Tobacco Chloroplasts

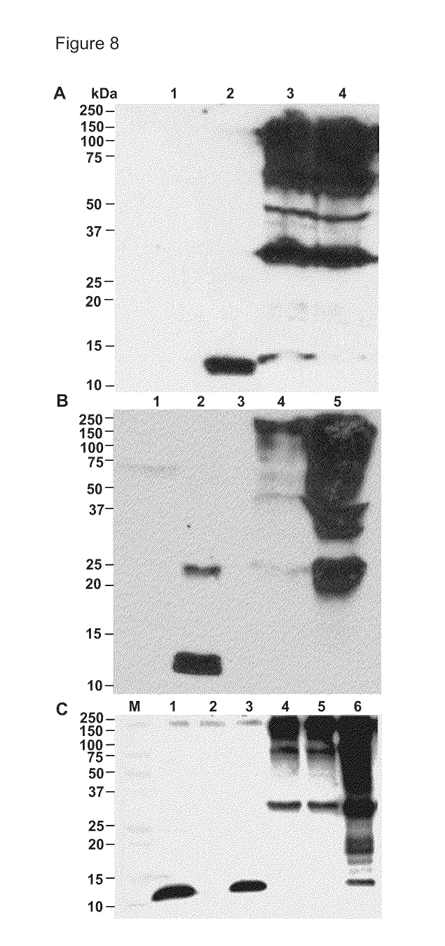

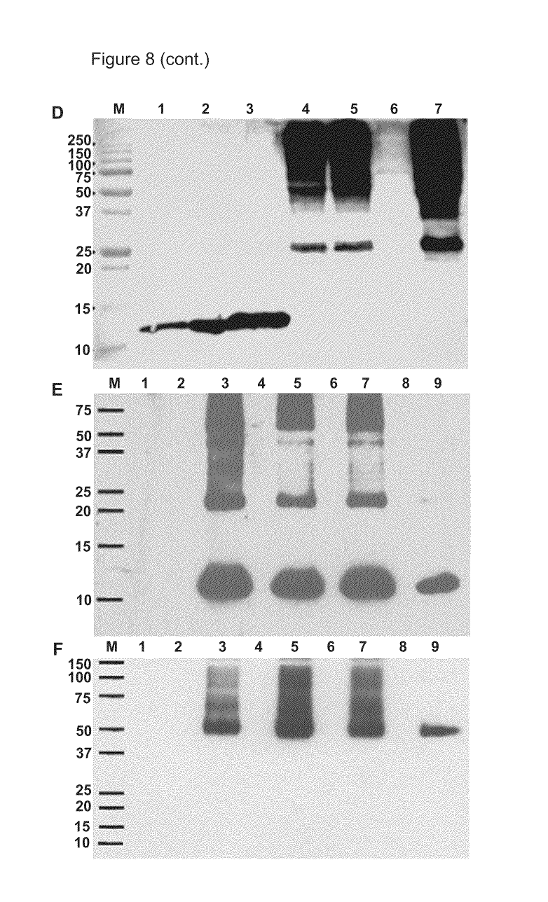

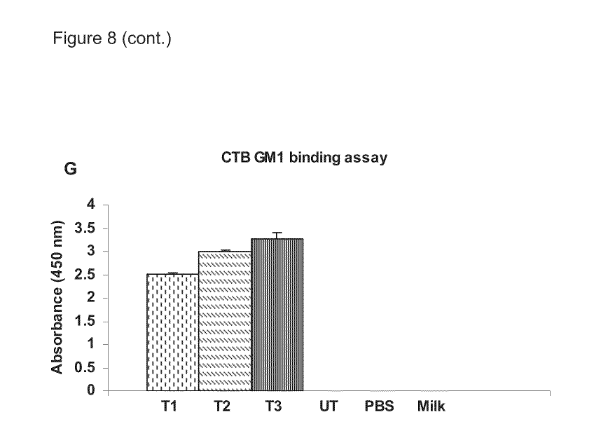

Immunoblots were performed with tobacco and lettuce transplastomic lines expressing CTB, CTB-AMA1 and CTB-MSP1 (FIGS. 8A-F). Immunodetection with CTB polyclonal antibody showed 11.5 kDa of the CTB monomer, 27.5 kDa monomer of CTB fused with AMA1 and a 23 kDa monomer of CTB fused with MSP1 (FIGS. 8A-F).The formation of dimers, trimers, tetramers and pentamers of the CTB, CTB-AMA1 and CTB-MSPlfusion proteins was observed in tobacco as well as in lettuce. The monomer, dimer and pentameric forms of CTB were observed in all transgenic lines under denatured condition and only the pentameric or larger forms were observed under non-reducing conditions (FIGS. 8E, F). Foreign proteins could be detected in the supernatant and pellet (FIGS. 8A, B). Therefore, the quantification of CTB, CTB-AMA1 and CTB-MSP1 was performed using homogenate.

ELISA was performed to quantify the chloroplast derived CTB, CTB-AMA1 and CTB-MSP1 antigens in the homogenate of lettuce and tobacco. A standard curve was obtained with the purified bacterial CTB. The CTB-AMA1 and CTB-MSP1 expression level of tobacco transplastomic lines in mature leaves reached up to 12.3% and 8% of the total soluble protein (TSP), respectively. In lettuce CTB-AMA1 and CTB-MSP1 protein expression level reached up to 9.4% and 4.8% of the TSP, respectively in mature leaves under the green-house growth conditions (Table 1). A gram of mature leaf yielded up to 3.33 mg and 1.56 mg of CTB-AMA1 fusion proteins in tobacco and lettuce respectively. A gram of mature leaf yielded up to 2.16 mg and 0.66 mg of CTB-MSP1 antigen in transformed tobacco and lettuce respectively.

TABLE 1
Quantification of vaccine antigens in transgenic plants.

| | | | | Percentage | Amount of |
|---|---|---|---|---|---|
| | | | Amount of | of | transgene |
| Transgenic | | TSP | transgene | transgene | protein in |
| tobacco | | μg | protein | protein in | per gram of |
| and lettuce | gene | μl⁻¹ | μg μl⁻¹ | TSP | leaf tissue |
| Nicotiana | CTB-ama1 | 9.0 | 1.11 | 12.3 | 3.33 mg |
| tabaccum | | | | | |
| Nicotiana | CTB-msp1 | 9.0 | 0.72 | 8.0 | 2.16 mg |
| tabaccum | | | | | |
| Lactuca | CTB-ama1 | 5.5 | 0.52 | 9.4 | 1.56 mg |
| sativa | | | | | |
| Lactuca | CTB-msp1 | 4.5 | 0.22 | 4.8 | 0.66 mg |
| sativa | | | | | |

Quantification of CTB-AMA1 and CTB-MSP1 protein in chloroplast transformed tobacco and lettuce by ELISA as described in materials and methods. Primary anti-rabbit CTB polyclonal antibody and secondary antibody HRP-conjugated donkey anti-rabbit at 1: 5000 were used to quantified CTB-fusion proteins (CTB-AMA1 and -MSP1).

B.4 G1% Binding of Chloroplast-Derived CTB

GM₁₋ganglioside has been shown to be the receptor for CTB protein in vivo and a pentameric structure is required for binding to GM₁ receptor. To investigate functionality of chloroplast-derived CTB, we performed GM₁ binding ELISA assay. As illustrated in FIG. 8G chloroplast-derived CTB is fully functional and binds to GM₁. These results confirm that the lettuce chloroplast derived CTB is properly folded to form pentamers, which is essential for GM₁₋ganglioside receptor binding.

B.5 Enrichment of Chloroplast-Derived Antigens

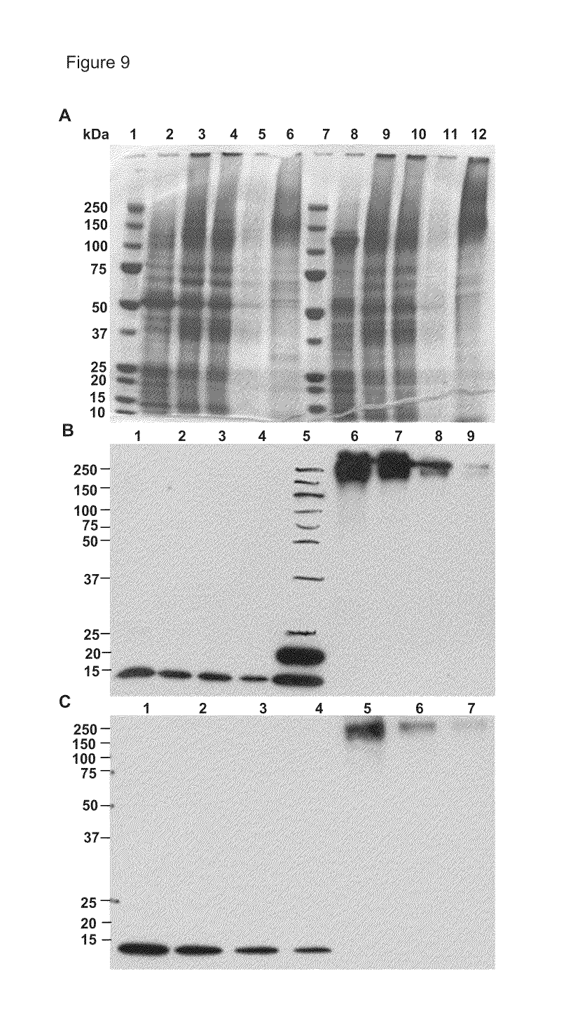

A crude extract of chloroplast-derived proteins was subjected to immobilized metal affinity chromatography by using the TALON Superflow Metal Affinity Resin and analysis followed. A NuP AGE Novex Bis-Tris gradient gel was used to increase the resolution of the enriched CTB-AMA1 protein. The gel was performed under reduced and non-reduced conditions. The large subunit of rubisco (55 kDa) is apparent in the untransformed, lysate, and flow through fractions under reduced and non-reduced conditions (FIG. 9A). In the wash fractions minimal number of proteins was observed. In the eluted CTB-AMA1 fraction, the monomer of 27.5 kDa in size is present under reduced conditions (Lane 6) and the pentameric form is present under both reduced (Lane 6) and non-reduced (Lane 12) conditions (FIG. 9A). It should be noted that the pentameric form is the dominant form and this should facilitate GM1 binding. An immunoblot probed with anti-CTB antibody was conducted to confirm the presence of the CTB-malarial proteins after talon enrichment. An immunoblot with known concentrations of CTB protein and different concentrations of the enriched fractions were probed with anti-CTB antibody. Quantitation of the enriched CTB-malarial proteins on immunoblots was analyzed by densitometry. Linearity of the standard curve assisted in the estimation of the enriched samples in the same blot (FIG. 9B, C). The efficiency of the talon enrichment was determined to be 90% and 73% in CTB-AMA1 and CTB MSP1, respectively.

B.6 Sera of Immunized Mice Protects CHO Cells from Dehydration After CT Treatment

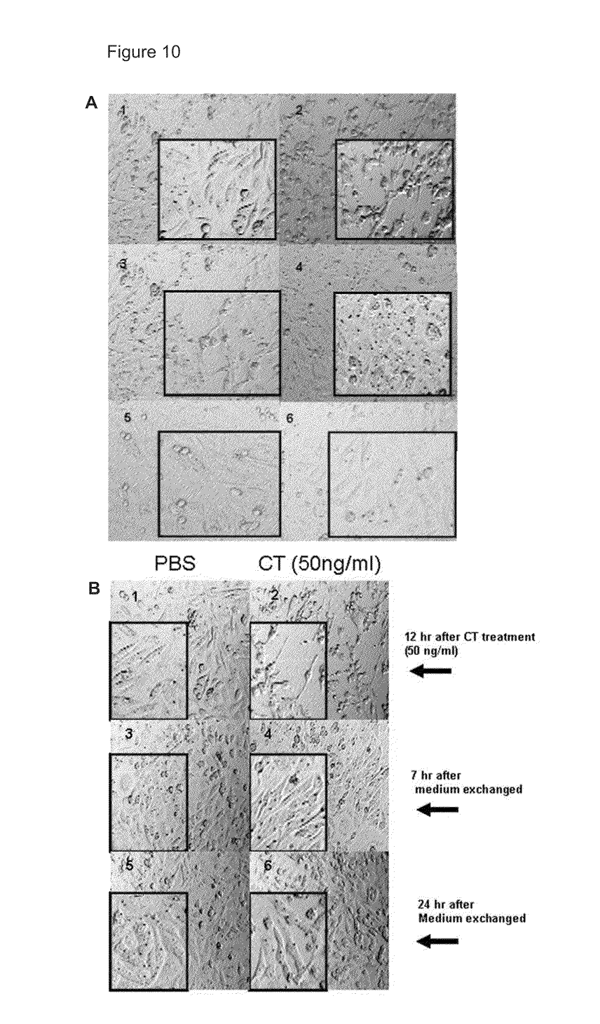

In order to examine the biological activity of antibodies induced by oral or subcutaneous administration of CTB, CHO cell elongation assay was performed with pooled sera of vaccinated and control mice as described elsewhere [34]. Our data show that sera of immunized mice, regardless of route of immunization, protected morphological changes (elongation) due to dehydration in CHO cell culture (FIG. 10A). In contrast, CHO cells treated with sera of unimmunized control mice showed massive elongation. When cell viability was checked 12 hr after CT treatment, using trypan blue exclusion method, we were unable to find cell death (>5%) in all conditions tested, including the CT treated cells (positive controls). Based on this observation we reasoned that morphological changes in CHO cells is transient and can be reversed by toxin removal. To investigate this hypothesis, we replaced 50% of the cell culture supernatant containing CT with fresh media and examined cell morphology after 7, 12 and 24 hrs. As shown in FIG. 10B, almost 80% of the CHO cells recovered after 7 hrs and there was very little morphological difference between PBS treated (negative control) and CT treated cells after 12 hrs. CHO cells were indistinguishable with control PBS treated after 24 hrs (FIG. 10B). These data suggest that dehydration of CHO cells because of CT treatment is a transient state and cells can be reversed by CT removal within 7-24 hrs. To the best of our knowledge, reversibility of dehydration has not yet been described elsewhere.

B.7 Mechanism of Protection from Cholera Toxin Challenge

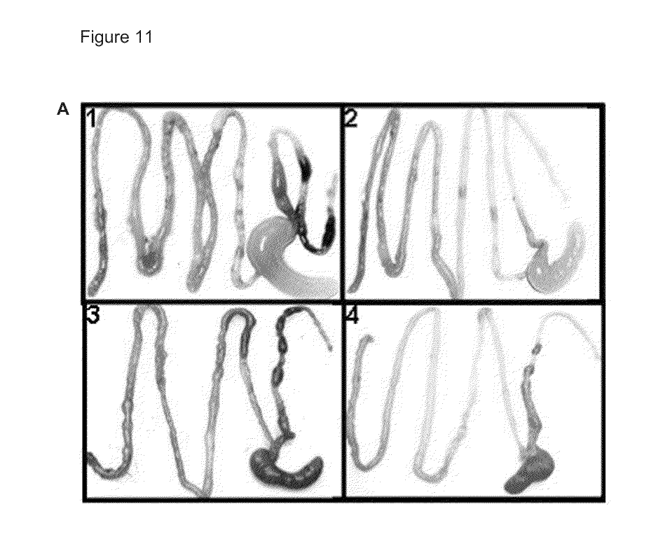

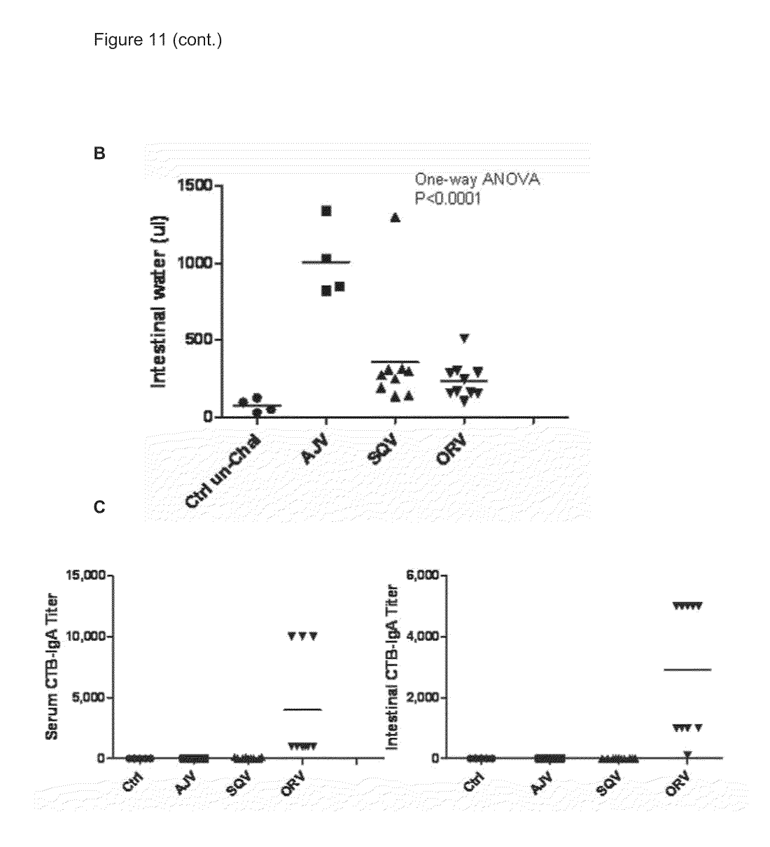

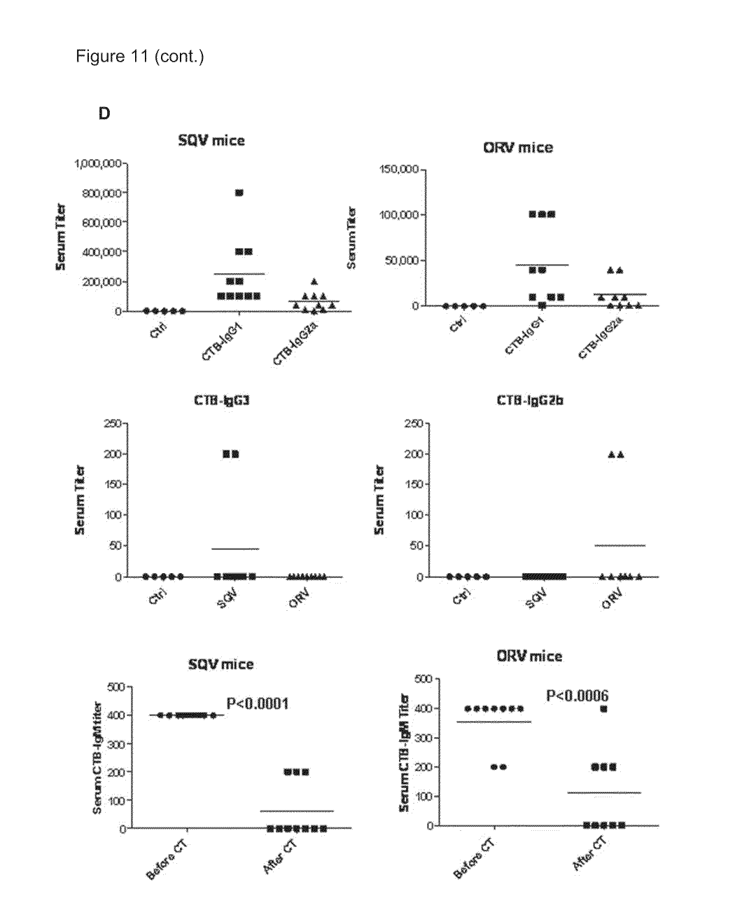

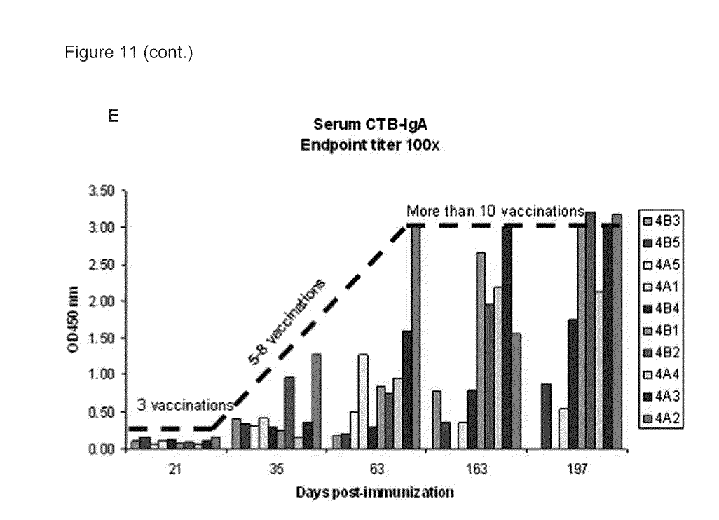

A broad range of CT concentration has been used by investigators. BALB/c mice immunized with adjuvant (AJV), subcutaneous (SQV) or orally immunized with plant cells expressing CTB (ORV-CTB) or untransformed leaves (ORV-UT) were challenged with cholera toxin as described in the materials and methods section. We found a significant association between the volume of intestinal water retention in SQV and ORV-CTB mice and subcutaneous or oral immunization with CTB (FIG. 11A, B). However, there was no significant difference in intestinal water content between SQV and ORV-CTB mice. Control mice immunized with adjuvant (AJV) or gavaged with untransformed leaf developed severe diarrhea (FIG. 11A, B).

Our antigen-specific ELISA data showed presence of serum and intestinal CTB-IgA in ORV-CTB mice but not in SQV, AJV and/or in control mice suggesting a direct correlation between IgA and protection in orally vaccinated mice (FIG. 11C). It should be noted that IgA titers repeatedly and reproducibly observed in ORV-CTB mice are much higher than those reported in previous studies. In contrast, in SQV mice that were protected from CT challenge, we were unable to detect any CTB-IgA in serum and/or in intestine by ELISA. To investigate the mechanism of protection observed in SQV mice, we screened a broad range of antigen-specific immunoglobulins by ELISA including CTB-IgG1, -IgG2a, -IgG2b, -IgG3 and -IgM in the sera of vaccinated and control mice. Our data show that only CTB-IgG1 and no other tested immunoglobulin in this study conferred protection in SQV mice (FIG. 11D). Again, it should be noted that the mean IgG1 titer observed in SQV mice was about 250,000. Screening of the same profile of immunoglobulins in the sera of ORV mice showed comparable pattern of expression with SQV mice as shown in FIG. 11D, in addition to intestinal and serum IgA, suggesting that oral vaccination provides both mucosal and systemic immune response in contrast to subcutaneous immunization that provides only systemic immune response. Furthermore, we screened CTB-IgG1, -IgG2a, -IgG2b, -IgG3 and -IgM in the sera of vaccinated mice before and after CT challenged and our data show that only CTB-IgM level significantly changed after CT challenge (FIG. 11D).

We also screened expression of IL-4 (Th2), IL-10 (Th2), IL-2 (Th1), IFNγ (Th1) and IL-17A (Th17) by ELISA in the sera of our experimental and control groups. Our data show that expression of IFNγ was detectable in 70% (7 of 10 mice), 16.6% (1 of 6 mouse) and 10% (1 of 10 mice) of control, SQV and ORV-CTB mice, respectively suggesting blocking of Th1 immune response in vaccinated mice. IL-17A is unlikely to play a role in this system because only one mouse in AJV and ORV-CTB groups were positive for this cytokine.

We also determined minimum number of vaccination to generate adequate antigen-specific antibody for effective protection from toxin challenge. As illustrated in FIG. 11E, it appears that a total number of 5 vaccinations are sufficient to reach to >90% immunity. Although subsequent boosters increased or decreased IgA titers in individual mice, all of them were protected from toxin challenge, despite 8-10 fold difference in IgA titers. This information is useful for generation of effective vaccination regiment with optimal number of boosters. Most of the currently used vaccines required 3-5 boosters (http://www.cdc.gov/).

B.8 Immunogenicity of Malarial Antigen MSP1

Female BALB/c mice were immunized orally (ORV) with transgenic-leaf materials expressing MSP1 or by subcutaneous injections (SQV) with enriched MSP1 bound to the adjuvant and sera was collected on days 21, 35, 63, 163, and 197-post immunization. The serum was tested for anti-PfMSP-1₁₉ antibody by capture ELISA. As shown in Table 2, both SQV and ORV mice generated significant amount of anti-MSP1-IgG1 antibody, although MSP-1 is not as highly immunogenic as CTB. More homogenous level of antibody titer was observed in ORV mice (1000-12,500) than SQV mice (1000-50,000). Four mice in groups 5 and 6 (5A1, 5B3, 6A1, 6B4) showed undetectable titers with MRA-49 PfMSP1₁₉protein (Table 2) but showed similar CTB titers with the other mice in the group. No antigen-specific antibody was detected in AJV and/or in WT gavaged control mice, confirming specificity of the generated antibody.

TABLE 2
Immunogenicity studied using MSP1 Protein in two different groups of mice.

| | MSP1-IgG1 | | MSP1-IgG1 | | MSP1-IgG1 |
|---|---|---|---|---|---|
| | Titers Bleed | MSP1-IgG1 | Titers Bleed | MSP1-IgG1 | Titers Bleed |
| Mouse # | #1 | Titers Bleed #2 | #3 | Titers Bleed #4 | #5 |
| 5A1 | 0 | 0 | 0 | 0 | 0 |
| 5A2 | 100 | 1000 | 25000 | 25000 | 50000 |
| 5A3 | 1000 | 1000 | 25000 | 25000 | 25000 |
| 5A4 | 100 | 1000 | 12500 | 25000 | 50000 |
| 5A5 | 0 | 1000 | 1000 | 1000 | 1000 |
| 5B1 | 0 | 0 | 250 | 1000 | 12500 |
| 5B2 | 100 | 1000 | 12500 | 25000 | 25000 |
| 5B3 | 0 | 0 | 0 | 0 | 0 |
| 5B4 | 0 | 1000 | 25000 | 25000 | 50000 |
| 5B5 | 0 | 0 | 1000 | 1000 | 12500 |
| 6A1 | 0 | 0 | 0 | 0 | 0 |
| 6A2 | 0 | 500 | 1000 | 12500 | 12500 |
| 6A3 | 100 | 250 | 1000 | 12500 | 12500 |
| 6A4 | 100 | 1000 | 1000 | 1000 | 1000 |
| 6A5 | 0 | 0 | 250 | 1000 | 1000 |
| 6B1 | 0 | 0 | 250 | 1000 | 1000 |
| 6B2 | 500 | 1000 | 12500 | 12500 | 12500 |
| 6B3 | 0 | 100 | 1000 | 1000 | 1000 |
| 6B4 | 0 | 0 | 0 | 0 | 0 |
| 6B5 | 0 | 250 | 1000 | 12500 | 12500 |

Detection of anti-MSP1₁₉ antibody in sera of mice from groups 5 and 6 collected from five different time points as described in the text. Sera of SQV and ORV-MSP1 mice were subjected to an antigen-specific MSP1-IgG1 ELISA.

B.9 Generated Antibody in Vaccinated Mice Cross-Reacted with Plasmodium Proteins

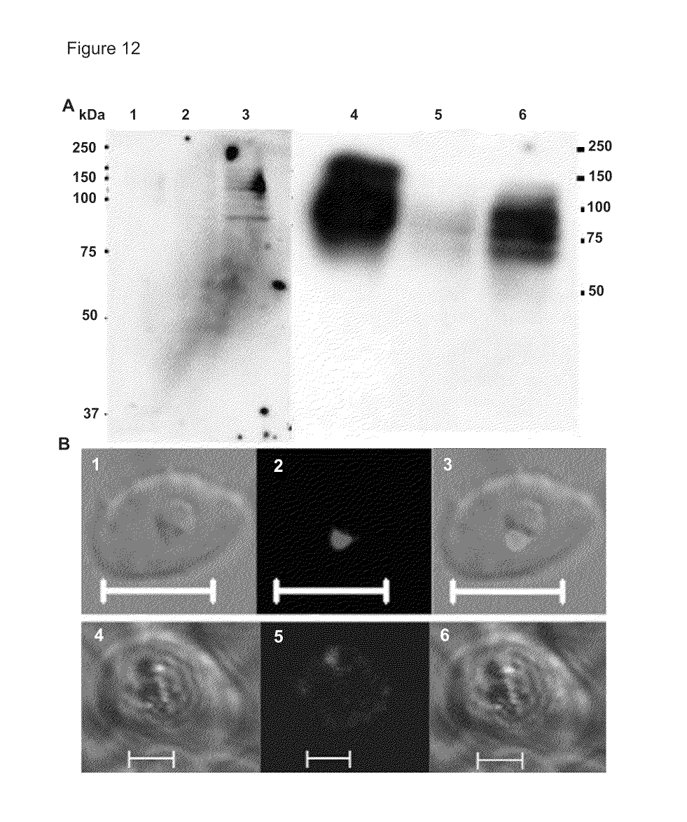

To determine whether the sera collected from mice immunized with chloroplast-derived CTB-malarial antigens recognized the native parasite proteins and native parasites, they were studied by immunoblots and immunofluorescence. Anti-AMA1 antibody in the sera recognized the schizont stage protein extracts with the presence of a 83-kDa polypeptide (FIG. 12A). The sera from immunized mice contained anti-MSP1 antibodies that recognized ring and schizont stage protein extracts with a 190-kDa polypeptide (FIG. 12A). Anti-AMA1 antibodies were found in the immunized sera because native parasites were stained in the apical end of the parasite (FIG. 12B) at the ring stage. Sera from mice immunized with the chloroplast-derived CTB-MSP 1 antigen stained schizonts indicating the presence of anti-MSP 1 antibodies (FIG. 12B).

B.11 Generated Antibody in Immunized Mice Inhibits Plasmodium Entry Into RBCs

In vitro parasite inhibition assays were performed to evaluate the ability of anti-MSP1 antibodies in inhibiting parasite entry into erythrocytes. The predominant stage found under microscopic examination was the ring stage. The average parasitemia for the blank control (no serum added) was determined to be 6.6% while the lowest parasitemia was observed in group with the highest MSP-1₁₉ titer (Table 3). The serum from the positive control (MRA-35 rabbit antiserum against purified from recombinant yeast, PfMSP1-19, 3D7) was used as positive control for 100% inhibition (Table 3). The remaining experimental groups displayed 85.8-105.8% inhibition when compared with the positive control (Table 3). Slightly lower inhibition observed in groups 7 and 8 was anticipated because the antigen doses were lowered by 50% to accommodate two antigens as opposed to 100% for single antigens. The control groups that did not receive chloroplast-derived CTB-malarial antigens resulted in 14.3-25.7% inhibition relative to the positive control (Table 3). There was good correlation between MSP1 titers and parasite inhibition. Mice with anti-MSP 1 antibody titers of 50,000 exhibited the highest inhibition in the parasite inhibition assay (Table 4), although mice with antibody titer of 1000 demonstrated quite effective parasite inhibition in vitro. Similar to variable CTB titers that conferred complete protection in CT challenged mice, MSP1 titers were variable but effective. As shown in tables 2-4, relative inhibition in vaccinated mice, regardless of the route of vaccination, was 86-117% (±15.5%), when compared with the positive control, while relative inhibition observed in the sham treated control groups was 14.3-25.7%. The relative inhibition with the sera of vaccinated mice was as effective as and/or better than the positive control obtained from NIH.

TABLE 3
Parasitemia assays and relative inhibition of parasite
in RBCs by sera of different groups of mice

| | | | Relative |
|---|---|---|---|
| Group | Parasitemia | Mean Parasitemia | Inhibition |
| No Ab | 6.6-6.7% | 6.6% | — |
| MRA-35 PfMSP1-19 | 2.5-3.5% | 3.1% | 100.0% |
| Group 1 | 5.9-6.6% | 6.1% | 14.3% |
| Group 2 | 5.5-6.2% | 5.8% | 22.8% |
| Group 3 | 2.8-3% | 2.9% | 105.8% |
| Group 4 | 2.4-3.3% | 3.0% | 102.8% |
| Group 5 | 2.4-2.6% | 2.5% | 117.2% |
| Group 6 | 2.6-3.6% | 3.2% | 97.2% |
| Group 7 | 2.7-3.9% | 3.3% | 94.3% |
| Group 8 | 3.3-3.8% | 3.6% | 85.8% |
| Group 9 | 5.6-5.8% | 5.7% | 25.7% |

Average parasitemia and relative inhibition was determined by in vitro parasite inhibition assay. The stage of parasite used was trophozoit-schizont and the hematocrit and parasitemia were adjusted to 2%. Control and experimental mouse sera were heat inactivated and incubated with uninfected RBCs overnight at 4° C. The mouse serum was added to the parasite culture at a final concentration of 20%. The cultures were incubated for 48 hours to allow for schizont rupture and merozoite invasion. Assays were preformed in duplicate and repeated at least three times. Parasitemia was determined and the relative percent of inhibition was calculated by using the formula described in materials and methods.

TABLE 4
Correlation between MSP1 sera titer and parasite
inhibition in different groups of mice.

| Group | MSP1₁₉ Titer | Parasitemia | Inhibition |
|---|---|---|---|
| No Ab (Control) | — | 6.6% | — |
| MRA-35 PfMSP1-19 | — | 3.1% | 53% |
| Group 1 | 0 | 6.1% | 7.6% |
| Group 2 | 0 | 5.8% | 12.1% |
| Mouse 5A4 (s.c.) | 50000 | 2.4% | 63.6% |
| Mouse 5B5 (s.c.) | 12500 | 2.7% | 59.1% |
| Mouse 6B3 (oral) | 1000 | 3.5% | 47% |
| Mouse 6B5 (oral) | 12500 | 3.0% | 54.5% |
| Group 9 | 0 | 5.7% | 13.6% |

Average parasitemia and inhibition of invasion for individual mice was determined by an in vitro parasite inhibition assay. Sera collected from mice with different MSP-1 titers were used for assays. Assays were preformed in duplicate and repeated at least three times. For microscopic analysis, blood smears were stained with Giemsa and the number of parasites per 900-1,100 RBCs was counted. Parasitemia was determined and the percent of inhibition was calculated by using the formula described in materials and methods.

B.12 Response of Cellular Components of the Immune System to CT Challenge

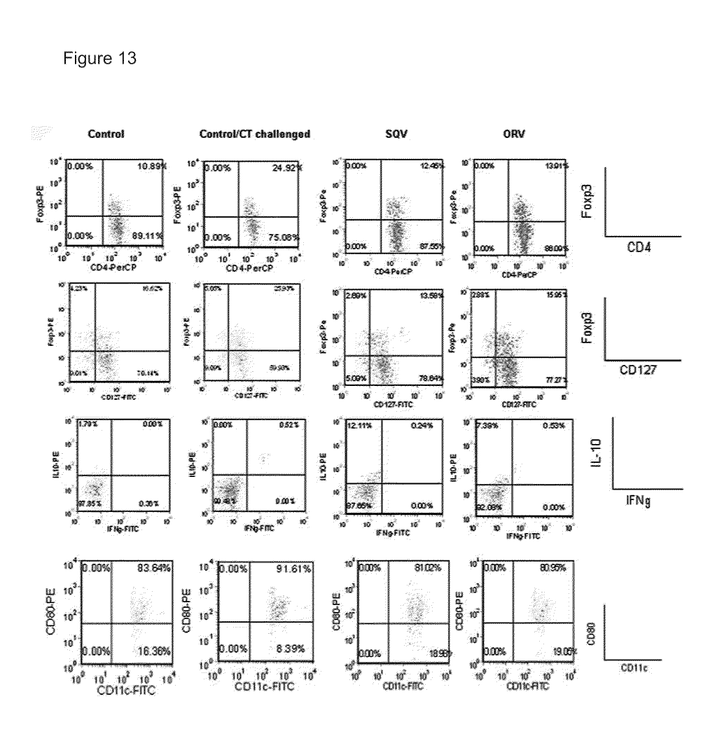

In order to study the impact of immunization on cellular components of the immune system, we measured expression of different markers associated with regulatory T-cells in fresh splenocytes obtained from controls (unvaccinated) and vaccinated mice after CT challenge. As shown in FIG. 13 (top row), CT challenge dramatically ameliorates numbers of CD4⁺Foxp3⁺ regulatory T-cell in unvaccinated control mice (increased from 11% to ˜25%). However, this effect was moderate in SQV (range from 7.2-12.5%) and ORV-CTB mice (range from 11.5-14%). As shown in FIG. 13, CT decreases expression of IL7Rα in unvaccinated mice but had marked upregulation in SQV and ORV-CTB mice. CT challenge eliminated CD4⁺IL10⁺ T-cells in unvaccinated control mice but significantly ameliorated this population in SQV and ORV-CTB mice, for ˜12% and 7.5%, respectively (FIG. 13). To this end, CT upregulated expression of co-stimulatory signal CD80 in CD11c⁺ splenic dendritic cells in unvaccinated control mice but this effect was neutral in vaccinated mice (FIG. 13). Collectively, these data suggest at least in part IL-10 expressing regulatory T-cells (Trl) but not Foxp3+ regulatory T-cells are crucial cellular components of the immune response in mice vaccinated with vaccine antigens.

C. Discussion

Production of an oral vaccine for major infectious diseases such as cholera and malaria with ease of administration and that does not require cold chain is an important need, especially in areas with limited access to cold storage or transportation. Considering that mucosal surface is the site for many gastrointestinal, respiratory and urogenital infections, developing an oral vaccine has great significance. For instance gastrointestinal infections caused by V. cholerae, Helicobacter pylori, Shigella spp and/or by rotaviruses, Entamoeba histolytica are major examples among many others. Many advantages of oral plant-derived vaccines to confer immunity against aforementioned infectious agents was discussed by us and others elsewhere.

Despite the recent increase in knowledge of genomics and proteomics of the malarial parasites, no licensed vaccine for the prevention of malarial disease is yet available. The need for a malarial vaccine is imperative because the global burden of the disease is increasing due to drug resistance, mosquito's resistance to insecticides, ineffective control measures, re-emergence of the disease, and increased tourism. There is a great need to create a low cost human malarial vaccine with the elimination of laborious and expensive purification techniques. Two leading blood stage malarial vaccine candidates, AMA1 and MSP1 were constructed in a fusion cassette with CTB. The CTB-malarial antigens were expressed in plants via the plastid genome at high levels.

It is believed that the study reported herein the longest cholera vaccine study reported so far in the plant-derived vaccine literature. Animals were boosted until 267 days and were challenged on day 303. Therefore, this study provides documentation on the longevity of mucosal and systemic immunity. This observation is significant in the light of recent reports on waning immunity against cholera. With the current cholera vaccine, immunity is lost in children within three years and adults are not fully protected. Although boosters beyond 5-8 did not significantly increase immunity levels, long-term protection was maintained. Considering the life span of BALB/c (˜2 years), this translates into protection up to 50% of mouse life span. Another interesting aspect of our study is the analysis of immunoglobulin in individual mice in each group whereas most previously reported studies used pooled sera for each group. Even though BALB/c mice are inbred strains, 8-10 fold variability observed within each group sheds new light on the correlation between immune titers and conferred protection. Such data should be valuable in prediction of protection in human clinical studies, amidst such variable immune response. The highest level of immune titers reported in this study may be due to high levels of CTB expression in chloroplasts and larger number of boosters given, although later boosters didn't significantly increase titers.

In the current study, we observed high level of CTB-IgA only in ORV-CTB mice but not in SQV or AJV or ORV-UT mice. In contrast, antigen presentation to the mucosal immune system via a non- receptor mediated delivery resulted in little or no local antigen-specific IgA. These data suggest that induction of intestinal IgA may require a receptor-mediated antigen presentation to the gut immune system and the antigen should be presented to the gut mucosal immune system and not to any other part of the systemic immune system. Further studies with antigens conjugated with and without CTB or other proteins that bind to intestinal receptors are necessary to understand the relationship between antigen presentation and production of IgA. Recently it has been shown that interaction of intestinal IgA with other locally generated cytokines such as TGFβ1, IL-10 and IL-4 will provide a unique microenvironment to educate DCs and subsequently educated DCs will imprint naïve T-cells [66] and imprinted T-cells secrete the same cytokine profile as previously antigen-experienced T-cells. None of SQV mice had detectable CTB-IgA; however, 89% of SQV mice were protected from CT challenge. Our data show that only serum CTB-IgG1 and not -IgG2a, -IgG2b, -IgG3 or -IgM confers immunity against CT challenge in SQV mice. Our data show that only CTB-IgM significantly decreased after CT challenge, while other members of the family remained the same. Our study has evaluated more immunoglobulins in response to delivery of plant-derived vaccine antigens than previous studies but further investigations are needed to fully understand this process.

Furthermore, our data from single-cell based studies suggest that CT increased numbers of Foxp3⁺ regulatory T-cells and co-stimulatory molecule CD80 in splenocytes in unvaccinated control mice but CT had little effect on this population in vaccinated mice. Increasing numbers of Foxp3 regulatory T-cells in unvaccinated mice is interesting because this population is the most effective arm of peripheral tolerance. Immediate consequences of higher numbers of Foxp3⁺ regulatory T-cell would be suppression of responding T-cell populations to CT. Because CT did not increase numbers of CD4⁺CD25^{+high} T-cells (data not shown), it appears that CT converts Foxp3⁻ CD25⁻CD4⁺ T-cells into Foxp3⁺ regulatory T-cells in the periphery. In agreement with our data, recently Sun et al. have reported increasing number of Ag-specific Foxp3⁺ regulatory T-cells by CTB and CTB plus CT, respectively (Sun J B, Raghavan S, Sjoling A, Lundin S, Holmgren J (2006) Oral tolerance induction with antigen conjugated to cholera toxin B subunit generates both Foxp3+CD25+ and Foxp3−CD25−CD4+ regulatory T cells. J Immunol 177:7634-7644). They have also demonstrated that intragasteric administration of OVA-CTB induced expansion of antigen-specific Foxp3⁺CD25⁺ regulatory T-cells, when compared with the sham treated control mice (Sun et al. 2006). In our study, CT induced upregulation of IL-10 expressing CD4⁺ T-cells, CTB-IgA and CTB-IgG1 in ORV and SQV, respectively, suggesting that vaccination regiment induced a Tr1/Th2 immune response and protected vaccinated mice against CT challenge. Upregulation of IL-7Rα⁺Foxp3⁻CD4⁺ T-cell in vaccinated mice after CT challenge is interesting because it has been reported that formation of Peyer's patches is dependent upon IL-7 receptor, TNF and TNF super family members. Further experiments are needed to address functional properties of IL-7R in plant-derived vaccines and immunity.

It has been reported that malarial antigens display poor immunogenicity even when used with adjuvants. Several strategies for increasing immunogenicity of malarial antigens include the use of different adjuvants, optimizing immunization protocols, using rabbits or monkeys for animal testing, fusing malarial antigen with viral or bacterial antigens, or constructing multivalent antigen chimeras. The expression of human malarial antigen, Plasmodium falciparum MCP-1 COOH-terminal region in tobacco plants via the nuclear genome has been reported earlier (Ghosh S, Malhotra P, Lalitha P V, Guha-Mukherjee S, Chauhan V S (2002) Expression of Plasmodium falciparum C-terminal region of merozoite surface protein (PfMSP1₁₉), a potential malaria vaccine candidate, in tobacco. Plant Science 162: 335-343) although expression level was extremely low (0.0035% tsp), 2300 fold lower expression than reported in our study. Furthermore, functionality of the plant-derived human malarial antigen was not investigated in this study. Recently, the rodent malarial antigen, P. yoelii codon-optimised MSP 4/5 was expressed in tobacco transgenic plants (Wang L, Webster D E, Campbell A E, Dry I B, Wesselingh S L et al. (2008) Immunogenicity of Plasmodium yoelii merozoite surface protein 4/5 produced in transgenic plants. Int J Parasitol 38:103-110) that showed modest expression level (0.25% tsp). Although this antigen induced specific antibody response, antibody titers were very low and failed to protect mice against parasite challenge. In our study, the human malarial antigens consisting of domain III of AMA1 and 19-kDa C-terminal fragment of MSP1 showed high levels of expression in both lettuce and tobacco chloroplasts (up to 12.3% tsp). Expression of AT-rich P. falciparum open reading frames is of particular advantage in the chloroplast expression system because the chloroplast genome is also AT-rich. The recombinant chimeric antigen was found to be highly immunogenic in mice. Although our in vitro inhibition assay provided evidence that the antibodies generated from immunized mice were effective in preventing parasite invasion of RBCs, a lethal parasite challenge could not be done. Evaluation of human vaccine antigens (from P. falciparum) in the rodent model system has major difficulties. Malaria challenge failed when mice are vaccinated with P. falciparum and challenged with P. berghei (Sun et al. 2006) suggesting that specific immunity is required for a specific parasite. One solution is to challenge immunized mice with the P. berghei/P. falciparum (Pb-PfM19) chimeric line that expresses the P. falciparum MSP-1(19). Parasite challenge was shown to be successful to protect mice when animals were passively immunized with anti-PfMSP1₄₂ antibody and then challenged with chimeric line of the chimeric line (Sachdeva S, Mohmmed A, Dasaradhi P V, Crabb B S, Katyal A et al. (2006) Immunogenicity and protective efficacy of Escherichia coli expressed Plasmodium falciparum merozoite surface protein-1(42) using human compatible adjuvants. Vaccine 24:2007-2016). However, this chimeric P. berghei line was not effective in control animals in our study and this strain has not yet been used in direct challenge studies.

In conclusion, this study for the first time demonstrates efficacy of an inexpensive vaccination method using transgenic plant-derived leaves to protect mice from two major infectious diseases, cholera and malaria. Currently, other than the rotavirus, there is no other example of oral vaccines in the US and the mucosal immune system has not been utilized to confer immunity against invading pathogens such as cholera and malaria. Oral polio vaccine was discontinued in the US because one in 2.4 million cases contracted polio from the live attenuated oral vaccine. However, such problems are not associated with subunit vaccines because only one or two antigens are used that are incapable of causing any disease. Therefore, it is important to understand and utilize the mucosal immune system for delivery of subunit vaccines. Bioencapsulation of vaccine antigens in plant cells provide an ideal low cost delivery system for large-scale distribution at times of crisis. In addition, oral delivery confers dual protection via systemic and mucosal immune system. High level and long-term protection observed against cholera toxin challenge in mice and against the malarial parasite in mice sera immunized with chloroplast-derived antigens, makes this system yet another new platform for advancing towards human clinical studies.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

What is claimed is:

1. A method for increasing an immune response in a subject simultaneously against cholera infection and malarial infection, comprising administering to the subject an immunizing amount of a composition comprising CTB-AMA1 polypeptide, wherein said CTB-AMA1 polypeptide is derived from a plastid transformed to express said CTB-AMA1 polypeptide.

2. A composition derived from a plant, said composition effective in increasing an immune response in a subject against Cholera and Malarial infection, said plant composition comprising a therapeutically effective amount of CTB-AMA1 polypeptide and rubisco.

3. The composition of claim 2, wherein said plant comprises a plastid transformed with a stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for said CTB-AMA1 polypeptide, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

4. A method for increasing an immune response in a subject simultaneously against cholera infection and malarial infection, comprising administering to the subject an immunizing amount of a composition comprising CTB-MSP1 polypeptide, wherein said CTB-MSP1 polypeptide is derived from a plastid transformed to express said CTB-MSP1 polypeptide.

5. A composition derived from a plant, said composition effective in increasing an immune response in a subject against cholera and Malarial infection, said plant composition comprising a therapeutically effective amount of CTB-MSP1 polypeptide and rubisco.

6. The composition of claim 5, wherein said plant comprises a plastid transformed with a stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for said CTB-MSP1 polypeptide, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

7. The method of claim 1, wherein said subject is a human or nonhuman mammal.

8. The method of claim 4, wherein said subject is a human or nonhuman mammal.

9. A plant comprising a plastid transformed to express a CTB-AMA1 or CTB-MSP1 polypeptide.

10. The composition of claim 5 wherein said composition further comprises a therapeutically effective amount of CTB-AMA1.
